(19) United States
(12) Patent Application Publication
(10) Pub. No.: US 2006/0041013 A1
(43) Pub. Date: Feb. 23, 2006

(54) Alanosine formulations and methods of use

(76) Inventors: Jason Edward Brittain, El Cajon, CA (US); Joe Craig Franklin, San Diego, CA (US); Lorenzo M. Leoni, Lodrino (CH); Christina Carol Niemeyer, Poway, CA (US); Gary T. Elliott, San Diego, CA (US)

Correspondence Address: BIOTECHNOLOGY LAW GROUP, C/O PORTFOLIOIP, P.O. BOX 52050, MINNEAPOLIS, MN, 55402, US

(21) Appl. No.: 11/153,640

(22) Filed: Jun. 14, 2005

Related U.S. Application Data
(60) Provisional application No. 60/602,803, filed on Aug. 18, 2004.
(60) Provisional application No. 60/613,779, filed on Sep. 27, 2004.
(60) Provisional application No. 60/673,493, filed on Apr. 20, 2005.

(51) Int. Cl. A61K 31/21

(52) U.S. Cl. 514/509

(57) ABSTRACT

Stable liquid formulations of the anti-tumor agent L-alanosine are described. These formulations preferably comprise L-alanosine in an aqueous environment having a basic pH, preferably in the range of about pH 8-9. The alanosine formulations and compositions disclosed herein can be used for various purposes, including the treatment of various cancers, particularly those that are deficient in methylthioadenosine phophorylase (MTAP) enzymatic activity. Also described are methods for the treatment of diseases susceptible to treatment with alanosine, e.g., certain cancers, particularly those characterized by tumor cells that are MTAP deficient, wherein a patient is administered L-alanosine, alone or as part of a combination therapy with a second chemotherapeutic agent.

### RELATED APPLICATIONS

This application claims the benefit of and priority to each of co-owned U.S. provisional patent application No. 60/602,803, filed 18 Aug. 2004, U.S. provisional patent application No. 60/613,779, filed 27 Sep. 2004, and U.S. provisional patent application No. 60/673,493, filed 20 Apr. 2005, each of the same title as this application, and each of which is hereby incorporated by reference in its entirety for all purposes.

### TECHNICAL FIELD

This invention concerns compounds having pharmaceutical utility. Specifically, it concerns pharmaceutical formulations of alanosine, and methods of using alanosine to effect desired therapeutic outcomes.

### BACKGROUND OF THE INVENTION

1. Introduction

The following description includes information that may be useful in understanding the present invention. It is not an admission that any such information is prior art, or relevant, to the presently claimed inventions, or that any publication specifically or implicitly referenced is prior art.

2. Background

Alanosine is an antibiotic compound discovered in the 1960's. See U.S. Pat. No. 3,676,490; Thiemann and Beretta (1966), J. Antibiot., vol. 19A:155; Coronelli, et al. (1966), Farmaco. Ed. Sci., vol. 21:269. Alanosine was initially obtained by fermenting a bacterium later identified as Streptomyces alanosinicus (A.T.C.C. accession no. 15710). Alanosine, an analog of the amino acid aspartic acid commonly found in nature in proteins, was the first natural product found to have a N-nitrosohydroxylamino group on an aliphatic chain. The compound has the chemical formula C₃H₇N₃O₄, and has a molecular weight of 149.1. The bacterial antibiotic compound has the structural formula:

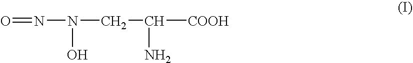

The naturally occurring L-isomer of alanosine, L(−)-2-amino-3-nitrosohydroxylaminopropionic acid, is slightly soluble in water, and is practically insoluble in common organic solvents. Because alanosine has two acid groups, it can form both neutral and acid salts. Alanosine reportedly gives salts with alkali and alkaline earth metals, and with inorganic and organic basic substances. In mice, the natural compound has very low toxicity, with an LD₅₀ of 600 mg/kg when administered intraperitoneally and 300 mg/kg when given intravenously. U.S. Pat. No. 3,676,490; Thiemann and Beretta, supra; Murthy, et al. (1966), Nature, vol. 211:1198.

In addition to its antibiotic effects, L-alanosine has also been investigated as a potential chemotherapeutic agent for the treatment of cancer, most recently in cancers wherein the cells are deficient in the enzyme methylthioadenosine phophorylase (MTAP) that, in normal mammalian cells, catalyzes the cleavage of methylthioadenosine (MTA) into adenine and methylthioribose-1-P (MTR-1-P). See U.S. Pat. Nos. 5,840,505 and 6,214,571. MTR-1-P is a substrate for metabolic synthesis of the amino acid methionine, one of the 20 naturally occurring amino acids used in protein biosynthesis. Adenine is salvaged into a cellular pool of adenosine 5′-monophosphate (AMP), from which cells derive adenosine 5′-triphosphate (ATP) for metabolic energy and 2′-deoxyadenosine-5′-triphosphate (dATP) for DNA synthesis. Thus, cells that lack MTAP must rely on other pathways or sources to produce methionine and adenine. Methionine can be obtained from food, and thus its biosynthesis is not essential. Adenine, on the other hand, is biosynthesized and, in the absence of MTAP, it is obtained by the action of the enzyme adenylosuccinate synthetase (ASS). ASS converts inosine 5′-monophosphate (IMP) to AMP. Interestingly, L-alanosine inhibits ASS activity. Thus, in cells that already lack MTAP activity, L-alanosine inhibition of ASS depletes those cells of AMP and ATP (in the absence of adenine).

Many clinical studies of the therapeutic efficacy of L-alanosine in human cancer patients, however, have been disappointing. See, e.g., Tyagi and Cooney (1984), Adv. Pharmacol. Chemotherapy, vol. 20:69-120; Creagan, et al. (1984), Adv. J. Clin. Oncol., vol. 7:543-544; VonHoff, et al. (1991), Invest. New Drugs, vol. 9:87-88; Creagan, et al. (1993), Cancer, vol. 52:615-618. Such disappointing results led to the eventual cessation of the clinical evaluation of L-alanosine as a potential anti-cancer agent. Later, investigators hypothesized that the studies were disappointing due in large part to the inability to identify patients whose cancers were homozygous for the defective MTAP allele and thus were likely to respond to L-alanosine treatment.

The inability to identify likely responders to L-alanosine treatment was recently overcome by the development of sensitive assays, e.g., nucleic acid amplification-based assays and immunohistochemical assays, which enable the detection of cancer cells that are homozygous for MTAP deficiency. See, e.g., U.S. Pat. No. 6,214,571. This advance, however, while important to the development of L-alanosine as an anti-tumor agent, has served to highlight other problems associated with the human clinical use of L-alanosine, including formulations and dosing regimens. Briefly, due to the low water solubility of the active ingredient (i.e., L-alanosine) and its poor stability in aqueous solutions of about pH 7, all existing L-alanosine formulations utilize a lyophilized product that must be reconstituted just prior to use. Further, the use of lyophilization fill-finish processes greatly increases the manufacturing costs and limits lot size. The requirement for reconstitution complicates administration and adds the requirement of a separate, suitable diluent.

Given the issues concerning L-alanosine formulations, the need clearly exists for improved L-alanosine compositions.

3. Definitions

Before describing the instant invention in detail, several terms used in the context of the present invention will be defined. In addition to these terms, others are defined elsewhere in the specification, as necessary. Unless otherwise expressly defined herein, terms of art used in this specification will have their art-recognized meanings.

As used herein, “alanosine” generally refers to L-alanosine (and its active metabolite, L-alanosinyl AICOR), unless otherwise stated or indicated by context. “D-alanosine” refers to the D-isomer of alanosine. A composition comprises “substantially all” of the D- or L-form of alanosine when the D- or L-form comprises at least about 90%, and preferably at least about 95%, 99%, and 99.9%, of the particular composition on a weight basis. A composition comprises a “mixture” of the D- and L-forms of alanosine when each isomer represents at least about 10% of the alanosine present in the composition on a weight basis. An alanosine molecule can be prepared as an acid salt or as a base salt, as well as in free acid or free base forms. In solution, alanosine molecules typically exist as zwitterions, wherein counter ions are provided by the solvent molecules themselves, or from other ions dissolved or suspended in the solvent.

An “alanosine analog” or “alanosine derivative” refers to a synthetic (i.e., non-naturally occurring) molecule derived from an alanosine isomer that is capable of inhibiting the enzymatic activity of ASS at least 10% as well as L-alanosine, as measured on mole-to-mole or number of molecules to number of molecules basis using the same assay, preferably by at least about 50%, 60%, 70%, 80%, 90%, 100%, or more as compared to a control reaction that does not contain an ASS inhibitor. The term “alanosine derivative” also refers to metabolites of alanosine that result following administration of the compound, as well as to prodrugs.

The term “amino acid” denotes a molecule or residue thereof containing an amino group and a carboxylic acid group. Amino acids can be naturally occurring and non-naturally occurring amino acids, as well as any modified amino acid that may be synthesized or, alternatively, obtained from a natural source.

A “degradation product” refers to a chemical that results from the chemical breakdown of a precursor chemical. In the context of the invention, a “degradation product” of alanosine refers to a one or more chemicals that result from the chemical breakdown of an alanosine molecule. The conversion of a D- or L-alanosine molecule to the other stereoisomer does not constitute a chemical breakdown, but rather the interconversion of one stereoisomeric form to another form. As will be appreciated, over time a some percentage of a population of homogenous L- or D-alanosine may undergo such an interconversion, such that the resulting population of alanosine molecules contains a portion of L-alanosine molecules and a portion D-alanosine molecules. The parameters affecting the rate and extent of interconversion between stereoisomeric forms will depend on many factors, including pH of the solution, storage temperature, etc. Different stereoisomers can be distinguished, for example, by including HPLC.

A “pH-insensitive container” refers to any container suitable for storage of a liquid pharmaceutical composition for more than one month under standard conditions, after which time the composition remains suitable for human administration. Such containers are typically comprised of materials that are resistant to appreciable breakdown when filled with a solution having a pH higher than pH 7.0 under the storage conditions specified. Materials useful in this regard include glass and plastics, for example, polypropylene.

The term “pharmaceutically acceptable salt” refers to salts which retain the biological effectiveness and properties of the compounds of this invention and which are not biologically or otherwise undesirable. In many cases, the compounds of this invention are capable of forming acid and/or base salts by virtue of the presence of amino and/or carboxyl groups or groups similar thereto. Pharmaceutically acceptable acid addition salts may be prepared from inorganic and organic acids, while pharmaceutically acceptable base addition salts can be prepared from inorganic and organic bases. For a review of pharmaceutically acceptable salts see Berge, et al. ((1977) J. Pharm. Sci., vol. 66, 1).

In the context of this invention, a “liquid composition” refers to one that, in its filled and finished form as provided from a manufacturer to an end user (e.g., a doctor or nurse), is a liquid or solution, as opposed to a solid. Here, “solid” refers to compositions that are not liquids or solutions. For example, such solids include dried compositions prepared by lyophilization, freeze-drying, precipitation, and similar procedures.

The terms “MTAP deficient”, “MTAP deficiency”, and the like refer to cells in which MTAP expression and/or activity is substantially reduced, even eliminated. For example, MTAP deficient cells include certain cancer cells that have undergone genetic mutations that render the cells MTAP deficient. Here, “substantially reduced” means that MTAP expression is insufficient to replenish the adenine pool in cells treated with a therapeutic amount of L-alanosine. Such reduction is typically at least a 50% reduction in the level of MTAP expression in the cell, as compared with a normal cell of the same lineage, i.e., a cell of the same type that is not diseased or otherwise exhibiting an MTAP deficiency. Preferably, MTAP expression is reduced 75%, 80%, 85%, 90%, 95%, 99%, or more as compared to normal cells of the same type. Even more preferred are cells in which MTAP expression is not detectable by the assay described in U.S. Pat. No. 6,214,571 or another nucleic acid-based diagnostic method. Also preferred are cells in which MTAP expression is not detectable or greatly reduced when assayed by immunohistochemical detection methods.

The expression “non-toxic pharmaceutically acceptable salts” non-toxic salts formed with nontoxic, pharmaceutically acceptable inorganic or organic acids or inorganic or organic bases. For example, the salts include those derived from inorganic acids such as hydrochloric, hydrobromic, sulfuric, sulfamic, phosphoric, nitric, and the like, as well as salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, fumaric, methanesulfonic, and toluenesulfonic acid and the like. Salts also include those from inorganic bases, such as ammonia, sodium hydroxide, potassium hydroxide, and hydrazine. Suitable organic bases include methylamine, ethylamine, propylamine, dimethylamine, diethylamine, diethanolamine, trimethylamine, triethylamine, triethanolamine, ethylenediamine, hydroxyethylamine, morpholine, piperazine, and guanidine.

A “patentable” composition, process, machine, or article of manufacture according to the invention means that the subject matter satisfies all statutory requirements for patentability at the time the analysis is performed. For example, with regard to novelty, non-obviousness, or the like, if later investigation reveals that one or more claims encompass one or more embodiments that would negate novelty, non-obviousness, etc., the claim(s), being limited by definition to “patentable” embodiments, specifically exclude the unpatentable embodiment(s). Also, the claims appended hereto are to be interpreted both to provide the broadest reasonable scope, as well as to preserve their validity. Furthermore, if one or more of the statutory requirements for patentability are amended or if the standards change for assessing whether a particular statutory requirement for patentability is satisfied from the time this application is filed or issues as a patent to a time the validity of one or more of the appended claims is questioned, the claims are to be interpreted in a way that (1) preserves their validity and (2) provides the broadest reasonable interpretation under the circumstances.

In the context of the liquid compositions of the invention, the term “stable” refers to the substantial lack of degradation or inactivation of the active ingredient species (e.g., L-alanosine) in the composition over time, preferably over more than 1, 6, 12, 24, or more months. Here, “substantial lack of degradation”, “substantial lack of inactivation”, and the like means that the population of molecules comprising the active species remains substantially intact and active such that the active ingredient meets its minimum specific activity specifications such that upon administration the composition provides the desired therapeutic benefit. In general, the active ingredient will remain at least about 90% of the molecules of the active ingredient in the composition will remain intact and active. Preferably, more than 95% of the molecules will remain intact and active. More preferably, at least about 98% of molecules of the active ingredient will remain intact and active, even more preferably, at least about 99% of the active ingredient will remain intact and active over the stated period. The extent of product degradation can be assessed using any suitable technique, including HPLC, gas chromatography, liquid chromatography, and mass spectrometry.

“Standard conditions” refers to storage conditions typically found in a pharmacy in a major hospital in a U.S. city. Minimally, these conditions refer to an ambient temperature of about 18-25° C. (i.e., no refrigeration), preferably 20-25° C. and even more preferably 25±2° C. and other environmental conditions suitable for long-term human presence. “Refrigeration” refers to storage conditions at a temperature of about 10° C. to about −2° C., preferably 5° C.±3° C.

A “therapeutically effective amount” refers to an amount of an active ingredient, e.g., alanosine, sufficient to effect treatment when administered to a subject in need of such treatment. In the context of cancer treatment, a “therapeutically effective amount” of alanosine is one which produces an objective tumor response in evaluable patients, where tumor response is a cessation or regression in growth determined against clinically accepted standards (see, e.g., Eagan, et al. (1979), Cancer, vol. 44:1125-1128, and the publicly available reports of parameters applied in the clinical trials performed under IND#14,247 (Food and Drug Administration)). With reference to these standards, determination of therapeutically effective dosages of, for example, L-alanosine, may be readily made by those of ordinary skill in the art. Of course, the therapeutically effective amount will vary depending upon the particular subject and condition being treated, the weight and age of the subject, the severity of the disease condition, the particular compound chosen, the dosing regimen to be followed, timing of administration, the manner of administration and the like, all of which can readily be determined by one of ordinary skill in the art.

The term “treatment” or “treating” means any treatment of a disease or disorder, including preventing or protecting against the disease or disorder (that is, causing the clinical symptoms not to develop); inhibiting the disease or disorder (i.e., arresting or suppressing the development of clinical symptoms; and/or relieving the disease or disorder (i.e., causing the regression of clinical symptoms). As will be appreciated, it is not always possible to distinguish between “preventing” and “suppressing” a disease or disorder since the ultimate inductive event or events may be unknown or latent. Accordingly, the term “prophylaxis” will be understood to constitute a type of “treatment” that encompasses both “preventing” and “suppressing”. The term “protection” thus includes “prophylaxis”.

### SUMMARY OF THE INVENTION

It is an object of this invention to provide patentable compositions comprising pharmaceutically acceptable formulations of alanosine in liquid form. Another object of the invention concerns methods of using the compositions of the invention to treat disease, including cancer, particularly cancers characterized as MTAP deficient, in humans and other mammals. Yet another object of the invention relates to the use of alanosine in combination with one or more other therapeutic agents.

Thus, one aspect of the invention concerns patentable liquid compositions comprising alanosine (or an alanosine analog) in solution, wherein the alanosine (or an alanosine analog) is stable for at least about one month, preferably at least about six months, even more preferably at least about twelve, eighteen, twenty-four months or more, when stored as a liquid when stored under standard conditions. Stability of the compositions of the invention can be further increased by refrigerated storage or freezing. In preferred embodiments that comprise alanosine, the alanosine is substantially all L-alanosine. In other embodiments, the alanosine is substantially all D-alanosine, while in other embodiments, the alanosine comprises a mixture of L-alanosine and D-alanosine. In particularly preferred embodiments, the alanosine is prepared from a pharmaceutically acceptable salt of alanosine. Such salts include those comprised of L-alanosine acid salt molecules and L-alanosine acid base molecules.

As alanosine, particularly L-alanosine, has antibiotic and anti-cancer properties, preferred compositions are formulated for administration to patients afflicted with a disease or disorder susceptible to treatment with alanosine or an analog thereof. When the patient to be treated is human, the composition is a pharmaceutically acceptable formulation. Such formulations typically contain the active ingredient, i.e., alanosine (or an alanosine analog) and a pharmaceutically acceptable carrier and/or a pharmaceutically acceptable excipient. When the patient is a non-human mammal (e.g., a bovine, canine, equine, feline, ovine, or porcine animal or a non-human primate), the composition is preferably a veterinarily acceptable formulation.

In preferred embodiments of this aspect, the alanosine (prepared either as an alanosine salt or acid) is dissolved in water, preferably water for injection, and the pH of the solution is at least about pH 7.5, preferably within the range of about pH 8 to about pH 12, even preferably about pH 8 to about pH 9. A particularly preferred pH is about pH 8.5. When the pH of the alanosine-containing solution is basic, the composition is preferably packaged in a pH-insensitive container. Preferred pH-insensitive containers are comprised of materials such as glass and plastic (e.g., polypropylene).

A related aspect concerns methods of making the compositions of the invention. In preferred embodiments of such methods, a stable aqueous formulation of alanosine (e.g., L-alanosine) is prepared by dissolving an alanosine salt or acid in water to make an alanosine solution. The pH of the alanosine solution is then adjusted to at least about pH 8, after which the pH-adjusted solution can be aliquotted into suitable containers. Such methods result in liquid compositions wherein the alanosine remains stable over a period of at least one month, preferably more than about six months, even more preferably more than about twelve months, and optimally greater than about twenty-four months even when stored under standard conditions. In other embodiments, where the pH of the solution is initially greater than that ultimately desired, e.g., when a di-sodium salt of L-alanosine is used as the starting material, the pH of the solution may be adjusted down using an appropriate acid.

Thus, a related aspect of the invention concerns kits containing alanosine. In general, such kits contain a composition comprising alanosine, preferably a pharmaceutically acceptable salt thereof, dissolved in a diluent or carrier, preferably a pharmaceutically acceptable diluent or carrier stored in a container. The container may be packaged in a box for storage and transport. For therapeutic applications, the box may further contain a package insert or the like describing how to use the composition in the container.

Another object of the invention concerns methods of treating patients having a disease susceptible to treatment with a composition containing alanosine, particularly L-alanosine, as described herein. Preferably, the patients are mammals, including humans, primates, and bovine, canine, equine, feline, ovine, and porcine animals. Preferably, the instant methods are used in the treatment of cancer, especially those wherein the cancerous cells are MTAP deficient. Representative examples of such cancers include acute lymphoblastic lymphoma, non-Hodgkin lymphoma, mesothelioma, glioma, non-small cell lung cancer (NSCLC), leukemia, bladder cancer, pancreatic cancer, soft tissue sarcoma, osteosarcoma, head and neck cancer, myxoid chondrosarcoma, and urothelial tumors.

Still another aspect of the invention concerns methods of addressing diseases and disorders amenable to treatment with alanosine in combination with another therapeutic agent, particularly chemotherapeutic agents. Examples of chemotherapeutic agents that can be used in combination with L-alanosine to effect treatment of various cancers include Taxotere®, 5-FU, vinorelbine, Alimta® (pemetrexed) gemcitabine, Tarceva® (erlotinib HCl), Iressa® (gefitinib), and Taxol® (paclitaxel).

### BRIEF DESCRIPTION OF THE DRAWINGS

FIG. 1 shows a titration curve of L-alanosine wherein 1N NaOH was used to adjust the pH.

FIG. 2 is a bar graph showing the relationship between total impurities as a function of increasing pH of L-alanosine samples stored at 80° C. for five days.

FIG. 3 is a table comparing over time (1, 2, 3, and 6 months) the stability of liquid L-alanosine formulations having different pH's and which had been stored at different temperatures. “CTM” refers to re-constituted L-alanosine samples prepared just prior to (“fresh”) or three or six days prior to analysis. Also, data for 4 and 5 month samples for aliquots of the pH 8.5 sample stored at 40° C. are presented below the table shown in this figure.

FIG. 4 has two panels, A and B. Panel A is an HPLC chromatogram of a sample of a liquid formulation of L-alanosine according to the invention (pH 8.5) after being stored at 5° C. for six months. Panel B is an HPLC chromatogram of an aliquot taken from a freshly reconstituted L-alanosine preparation prepared from a lyophilized composition containing the active ingredient.

FIG. 5 has two plots, A and B, showing the purity over time of three different aqueous L-alanosine formulations, pH 7.5, 8.5, or 9.0, stored at 50° C. (A) or 60° C. (B), as measured by HPLC.

FIG. 6 shows two HPLC chromatograms, A and B. Chromatogram A is an analysis of an aqueous L-alanosine formulation having a pH of 7.5, while chromatogram B is an analysis of an aqueous L-alanosine formulation having a pH of 8.5. In both cases, the samples were stored at 50° C. for 60 days prior to analysis.

FIG. 7 is an Arrhenius plot of purity showing results for each of three different aqueous L-alanosine formulations, pH 7.5, 8.5, or 9.0.

FIGS. 8A and 8B show graphical illustrations for rescuing ATP levels with an MTA analog in MTAP-positive but not in MTAP-negative cells treated with pemetrexed and L-alanosine.

FIGS. 9A-9C show synergistic effects of treating mesothelioma cells with L-alanosine and pemetrexed.

FIGS. 10A and 10B show synergistic effects of treating cells with L-alanosine and docetaxel.

FIGS. 11A and 11B show synergistic effects of treating cells with L-alanosine and 5-FU.

FIGS. 12A and 12B show in vivo effects of treating cells with L-alanosine and docetaxel. FIGS. 13A-C show the results of L-alanosine and paclitaxel monotherapy, as well a combination therapy of L-alanosine and paclitaxel, assessed in terms of percentage change in tumor volume (FIG. 13A), body weight (FIG. 13B), and days post-treatment initiation to attain a 10-fold increase in tumor volume (FIG. 13C). In FIG. 13A, an “*” indicates a day when the tumor volumes in mice treated using a combination therapy involving cycles of both L-alanosine and Taxol® administration were significantly different than in mice treated with paclitaxel alone (p<0.05, as measured using a non-parametric t-test).

### DETAILED DESCRIPTION

The present invention is based on the surprising and unexpected discovery that the anti-tumor compound alanosine, particularly compositions wherein the alanosine is substantially all of the L-isomer form, can be stably prepared and stored as a liquid composition over long periods of time. To achieve solubility and long-term in-solution stability of alanosine concentrations suitable for therapeutic use, it has been discovered that a basic aqueous solution, preferably having a pH of at least about 7.5, and preferably a pH of at least about 8 to about 12, is required. Compositions comprising such alanosine-containing solutions, and methods of making and using the same, are described in detail, below.

1. Preparation of Alanosine Compounds

An alanosine compound suitable for use in the invention may be obtained from any suitable source. For example, L-alanosine can be produced and purified from the medium of an S. alanosinicus culture, as described in U.S. Pat. No. 3,676,490. Alternatively, the compound may be generated by any suitable synthetic procedure known to those skilled in the art.

As those in the art will appreciate, alanosine compounds typically are amino acids, and thus contain an asymmetric center. As a result, alanosine and its analogs are capable of existing in stereoisomeric forms. All individual forms and mixtures thereof are included within the scope of the invention. The various isomers can be obtained by standard methods. For example, racemic mixtures can be separated into the individual stereoisomers by stereoselective synthesis, or by separation of the mixtures by fractional crystallization or chromatography techniques. In particular, individual enantiomers of alanosine may be prepared by resolution, such as by HPLC, of the corresponding racemate using a suitable chiral support or by fractional crystallization of the diastereoisomeric salts formed by reaction of the corresponding racemate with a suitable optically active acid or base, as appropriate. Individual enantiomers may also be obtained from a corresponding optically pure intermediate prepared by such a resolution method. These general principles are discussed in more detail by J. Jacques and A. Collet (“Enantiomers, Racemates and Resolutions”, Wiley, N.Y., 1981) and by W. Liu (“Handbook of Chiral Chemicals”, D. Ager (ed.), M. Dekker, N.Y., 1999; chapter 8).

The present invention also includes prodrugs that contain alanosine. Here, a prodrug is a compound that contains one or more functional groups that can be removed or modified in vivo to result in an alanosine molecule that can exhibit therapeutic utility in vivo.

2. Compositions

As described throughout this specification, the compounds of the invention are useful as therapeutic agents. The compounds will generally be formulated so as to be amenable to administration to a subject by the chosen route. Thus, a further aspect of this invention concerns pharmaceutical compositions comprising alanosine or an alanosine analog or derivative, or a pharmaceutically acceptable salt, base, or prodrug thereof, and a carrier, particularly a pharmaceutically acceptable carrier.

It will be appreciated that alanosine compounds have both acidic and basic functional groups. Therefore, in addition to the uncharged form depicted in the general formula, they may exist as internal salts (zwitterions). Furthermore, they may form pharmaceutically acceptable salts with acids and bases. Such zwitterions and salts are included within the scope of the invention. Alanosine salts can be prepared in situ during the final isolation and purification of the compounds of the invention or separately by reacting a free base function with a suitable organic acid. For example, a pharmaceutically acceptable salt of an alanosine compound of the invention may be readily prepared by mixing together solutions of alanosine and the desired acid or base, as appropriate. Stoichiometric quantities of reagents are preferably employed in order to ensure completeness of reaction and maximum production of yields of the desired final product. The salt may precipitate from solution and be collected by filtration or may be recovered by evaporation of the solvent. Salts may also be prepared by ion exchange, such as by equilibrating a solution containing alanosine with an appropriate ion exchange resin. Ion exchange may also be used to convert one salt form, such as a salt with an acid or base that is not pharmaceutically acceptable, to another salt form. Such methods are generally well known in the art.

Suitable acid addition salts are formed from acids that form non-toxic salts. Pharmaceutically acceptable acid addition salts may be prepared from inorganic and organic acids. Inorganic acids useful for producing inorganic salts include hydrochloric acid, hydrobromic acid, hydroiodidic acid, sulfuric acid, nitric acid, phosphoric acid, and the like. Organic acids useful for deriving organic salts include acetic acid, aspartic acid, butyric acid, propionic acid, glutamic acid, glycolic acid, pyruvic acid, oxalic acid, malic acid, malonic acid, palmitic acid, pectinic acid, picric acid, succinic acid, maleic acid, fumaric acid, tartaric acid, citric acid, benzoic acid, cinnamic acid, lactic acid, mandelic acid, nicotinic acid, benzenesulphonic acid, methanesulfonic acid, ethanesulfonic acid, p-toluene-sulfonic acid, salicylic acid, succinic acid, tartric acid, and the like. Also, as will be appreciated, basic nitrogen-containing groups can be derivatized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; and arylalkyl halides such as benzyl and phenethyl bromides and others.

Alanosine also contains acidic groups are capable of forming base salts with various pharmaceutically acceptable cations, for example, in situ during the final isolation and purification of an alanosine compound. Examples of such salts include the alkali metal or alkaline earth metal salts. Suitable base salts are formed from bases that form non-toxic salts. Pharmaceutically acceptable base addition salts can be prepared from inorganic and organic bases. Salts derived from inorganic bases include by way of example only, sodium, potassium, lithium, aluminum, ammonium, calcium, zinc, and magnesium salts, with sodium and potassium salts being particularly preferred. Salts derived from organic bases include, but are not limited to, salts of primary, secondary and tertiary amines, such as atkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group. Other representative organic amines useful for the formation of base addition salts include ethylenediamine, ethanolamine, diethanolamine, piperidine, piperazine and the like.

The present invention also provides compositions, particularly pharmaceutical compositions, that comprise alanosine, an alanosine analog or derivative, or a salt thereof formulated together with one or more non-toxic acceptable carriers, preferably pharmaceutically acceptable carriers. In this regard, alanosine, alanosine analogs and derivatives, and their respective acid or base salts can be formulated into liquid, preferably aqueous, formulations for storage and administration, as opposed to dried formulations that must be reconstituted just prior to administration to a subject. Liquid pharmaceutically administrable compositions can, for example, be prepared by dissolving, dispersing, etc. alanosine and optional pharmaceutical adjuvants in an aqueous carrier. Aqueous carriers include water (particularly water for injection into humans), alcoholic/aqueous solutions, and emulsions and suspensions. Preferred pharmaceutically acceptable aqueous carriers include sterile buffered isotonic saline solutions. Parenteral vehicles include sodium chloride solution, Ringer's dextrose, dextrose, and sodium chloride, lactated Ringer's, or fixed oils. Intravenous vehicles include fluid and nutrient replenishers, electrolyte replenishers (such as those based on Ringer's dextrose), and the like. Preservatives and other additives may also be present, such as, for example, antimicrobials, antioxidants, chelating agents, and inert gases and the like. Non-aqueous solvents may also be included, although when included they preferably comprise less than about 50%, more preferably lass than about 25%, and even more preferably less about 10%, of the total solvent volume of the solution. Examples of non-aqueous solvents include propylene glycol, ethanol, polyethylene glycol, vegetable oils such as olive oil, and injectable organic esters such as ethyl oleate. The pharmaceutical and veterinary compositions of the invention are preferably formulated for parenteral injection.

If desired, the pharmaceutical composition to be administered may also contain minor amounts of nontoxic auxiliary substances such as wetting agents, emulsifying agents, or solubilizing agents, antioxidants, antimicrobials, pH buffering agents and the like, for example, sodium acetate, sodium citrate, cyclodextrin derivatives, sorbitan monolaurate, triethanolamine acetate, triethanolamine oleate, etc. Actual methods of preparing such dosage forms are known, or will be apparent, to those skilled in this art; for example, see Remington: The Science and Practice of Pharmacy, Mack Publishing Company, Easton, Pa., 20th Edition, 2000. The composition or formulation to be administered will, in any event, contain a quantity of the active compound in an amount effective to alleviate the symptoms of the subject being treated.

In one preferred embodiment of the invention, a stable liquid formulation of alanosine can be prepared by first making a solution comprising 20 mg/mL L-alanosine in water, pH 6.5. In order to produce a stable liquid formulation, the pH of the solution is then adjusted to be at least about 7.5, preferably in the range of about 8-12. A particularly preferred pH is about pH 8.5. Adjusting the pH is preferably accomplished by adding increments of a strong basic solution, for example, 5N NaOH. After adjusting the alanosine-containing composition to its desired pH, it can then be aliquotted into suitable containers, preferably into containers suited for the storage of pharmaceutical compositions (i.e., in each case, a pharmaceutically acceptable container). If the pH of the final composition is more than about pH 9, the composition is preferably packaged in a pH-insensitive container suited for the storage of pharmaceutical compositions. Preferred pH-insensitive containers of this type are typically comprised of materials such as glass, e.g., glass coated with Teflon® and plastic, for example, polypropylene. A particularly preferred container is a 20 mL Schott vial that can be suitably sealed, for example, with a gray bromobutyl stopper fixedly secured in the vial's neck by a suitable clamp.

In some embodiments of the invention, the stable liquid formulations of the invention are prepared from lyophilized alanosine preparations. In other embodiments, they are prepared immediately following synthesis and purification. Also, in some embodiments related to the combination therapy aspect of the invention, L-alanosine may be re-constituted from a lyophilized preparation just prior to use, while in other embodiments, the L-alanosine composition used in the combination therapy is a stable alanosine-containing liquid formulation according to the invention that has not been re-constituted from a powdered formulation just prior to use.

As will be appreciated, lyophilized L-alanosine can be produced by any suitable method. In one such method, a solution containing 500 mg of L-alanosine prepared in accordance with any of U.S. Pat. Nos. 3,676,490; 6,210,917; and/or U.S. Pat. No. 6,214,571 is aliquotted into vials, such that each vial contains 5 mL of a solution containing 20 mg/mL L-alanosine at about pH 7. Using conventional lyophilization equipment, the vials are placed on trays and positioned evenly in the freeze-drying chamber. Preferably, the shelves on which the trays are placed are pre-cooled to facilitate rapid freezing of the vial contents. Also, thermocouples are preferably placed in the lyophilization chamber at positions that enable sufficient monitoring to ensure consistent conditions throughout the freeze drier during the lyophilization process. Upon completion of vial loading, the chamber door is sealed. A standard lyophilization cycle proceeds as follows: Once the warmest thermocouple registers −45° C., a timing period (e.g., at least three hours) is initiated. At the end of the timing period, the chamber is evacuated. When sufficient chamber vacuum is achieved (e.g., below about 200 microns), the temperature of fluid circulating through the chamber is slowly raised to slightly above freezing (e.g., to +5° C. (±2° C.)), for example, over a period of several (e.g., 12 (±2 hr.)) hours. The temperature of the circulating fluid is then maintained at the designated temperature (e.g., +5° C. (±2° C.)) for a relatively short period (e.g., two hours). Thereafter, the temperature of the circulating fluid is a raised to room temperature (about 18-25° C. (±2° C.)) or slightly above (e.g., +30° C. (±2° C.)) over several hours (e.g., about 8 hr.). A slightly cooler terminal drying temperature (e.g., +27° C. (±2° C.) when +30° C. (±2° C.) is the initial higher temperature) is then achieved and maintained for a period sufficient to evaporate all residual moisture from the vials (e.g., 24 hours). After completion of the vacuum drying cycle, the chamber is sealed off from the vacuum pump, and the chamber is bled to atmospheric pressure using sterile, dry nitrogen gas, USP, which is preferably passed through a microbiological filter. After reaching atmospheric pressure, the door to the freeze-drying chamber is opened. Vials are then sealed using a suitable seal (e.g., a stopper and foil seal). For example, in one embodiment, the vials can be aseptically sealed by mechanically collapsing the shelves to seat stoppers placed in the neck of each vial. In another embodiment, after placing stoppers in necks of the vials, the vials are transferred under laminar flow conditions in a nitrogen environment to a bench where stoppers can be seated and sealed.

In another lyophilization embodiment, vials each containing 5 mL of a solution of 20 mg/mL L-alanosine are placed in a conventional freeze-drying machine. The temperature inside the chamber is dropped to −40° C. to freeze the aqueous drug-containing solutions. That temperature is maintained at atmospheric pressure for 5 hr. Thereafter, the chamber is warmed to a temperature of about −15° C. at a rate of about +0.1° C./min. and evacuated to a pressure of about 150 milliTorr (mT). After achieving the desired temperature and pressure, those conditions are maintained for about 100 min. The chamber is then warmed another 5° C. at the rate of about +0.1° C./min., which temperature is then maintained for another 100 min. The temperature of the chamber is then raised to a temperature of about +30° C., again at the rate of about +0.1° C./min., and then held at +30° C. for 30 hr. Thereafter, the temperature inside the chamber is cooled to about +20° C. at the rate of about +1° C./min. The terminal drying temperature of +20° C. is then maintained for about 20-40 hr., after which the chamber is brought to atmospheric pressure, again using a dry gas, e.g., nitrogen, which has preferably been passed through a microbiological filter. The vials can then be capped and sealed as desired. Lyophilized compositions can be stored without refrigeration until just prior to use, at which time they are re-constituted using any suitable diluent or re-constitution buffer (e.g., a saline (e.g., 0.9% NaCl, w/v) solution for injection). A particularly preferred final L-alanosine concentration is 20 mg/mL.

3. Applications

As described above, certain aspects of the invention relate to compositions that contain alanosine, particularly L-alanosine, which compositions are useful in the treatment of cancer in humans and other mammals (e.g., bovine, canine, equine, feline, ovine, and porcine animals), as well as other animals. Specifically, this invention enables the treatment of cells, e.g., cancer cells, with stable liquid formulations of alanosine, particularly L-alanosine, which inhibits de novo adenine synthesis in such cells by inhibiting ASS. In cells that lack the capability to salvage adenine from metabolism of methylthioadenosine (MTA), ASS inhibition deprives them of such essential molecules as adenosine 5′-triphosphate (ATP). As a result, the alanosine-containing compositions and methods of the invention are useful in the treatment of certain cancers, especially those that are MTAP deficient, alone or in conjunction with other chemotherapeutic agents.

It is also worth noting that a major obstacle in effective cancer therapy concerns the ability of cancer cells to develop broad-spectrum resistance to many cytotoxic drugs, including the vinca alkaloids (e.g., vinblastine), the anthracyclines (e.g., doxorubicin), the epipodophyllotoxins (e.g., etoposide), the taxanes (e.g., taxol), antibiotics (e.g., actinomycin D), antimicrotubule drugs (e.g., colchicine), protein synthesis inhibitors (e.g., puromycin), toxic peptides (e.g., valinomycin), topoisomerase inhibitors (e.g., topotecan), DNA intercalators (e.g., ethidium bromide), and anti-mitotics. This phenomenon, termed multiple drug resistance (MDR), occurs to varying degrees in most cancers. The cell surface phospho-glycoprotein, P-glycoprotein, is believed to be one of the proteins that mediate MDR by acting as an energy-dependent efflux pump that expels hydrophobic drugs from cells. The expression of P-glycoprotein is increased in many cancer cells. While P-glycoprotein's precise mechanism of action is not known, its function is known to be energy-dependent, and cells that employ it require greatly increased stores of ATP (as compared to normal cells). Thus, the synthesis and metabolic turnover of ATP increases in growing and/or metastasizing cancer cells having up-regulated P-glycoprotein expression. Inhibiting de novo adenine synthesis interferes with the production of ATP, particularly in MTAP deficient cells. Because MTAP deficient cells cannot salvage adenine through salvage pathways, cells treated with alanosine become starved of adenine (and, consequently, of ATP) and die.

Thus, the compositions of the invention can be used to treat diseases and disorders in which inhibition of ASS activity would be of therapeutic benefit. Such diseases include various forms of cancer, particularly those wherein the cells are MTAP deficient. Cancers whose cells may be characterized by a genetically-caused MTAP deficiency include lymphoblastic lymphoma, non-Hodgkin lymphoma, mesothelioma, glioma, non-small cell lung cancer, leukemia, bladder cancer, pancreatic cancer, soft tissue sarcoma, osteosarcoma, head and neck cancer, myxoid chondrosarcoma, and urothelial tumors.

The compounds of the present invention may be used alone or in combination with other therapeutic agents or other anti-cancer therapies (e.g., radiation, surgery, bone marrow transplantation, etc.), as well as to potentiate the effects of other therapies, including treatment with other chemotherapeutic agents. As will be appreciated, “combination therapy” and the like refer to a course of therapy that involves the administration of at least two different therapeutic agents. The agents may be delivered using the same therapeutic regimen or different regimens, depending on the active ingredients involved, the disease to be treated, the age and condition of the patient, etc. Moreover, when used in combination with another therapeutic agent, the administration of the two agents may be simultaneous or sequential. Simultaneous administration includes the administration of a single dosage form that comprises both agents, and the administration of the two agents in separate dosage forms at substantially the same time. Sequential administration includes the prior, concurrent, or subsequent administration of the two or more agents according to the same or different schedules, provided that there is an overlap in the periods during which the treatment is provided. Suitable agents with which alanosine can be co-administered include chemotherapeutic agents such as Taxotere®, Taxol® (paclitaxel), 5-FU, vinorelbine, Alimta® (pemetrexed) gemcitabine, Tarceva™ (erlotinib HCl), and Iressa® (gefitinib).

Clinical trials studying the anti-tumor effects and usage of purine synthesis inhibitors as chemotherapeutic agents provide information concerning dosing and toxicity parameters for such inhibitors. In primates, approximately 75% of L-alanosine is excreted in urine in about 24 hours, primarily as the nucleoside forms of L-alanosinyl-IMP and L-alanosinyl-AICOR. Clearance from plasma after intravenous administration in humans is biphasic, with t_{1/2}α=14 minutes and t_{1/2}β=99 minutes (where “t_{1/2}” is the half-life, and times (t) are approximate).

In some cases, alanosine toxicity has been dose-limiting in certain treatments. Toxicities have reportedly included hepatotoxicity, renal toxicity, stomatitis, esophagitis and, with lesser frequency, myelosuppression, headache, nausea, and hypo- or hypertension. Renal toxicity has been reported to occur with single bolus dosing above 4 g/m² body weight. Two pediatric patients who received higher doses of about 350 mg/m² body weight per day in separate doses reportedly suffered liver failure. Stomatitis and esophagitis were reported to have occurred after multiple bolus dosing. In one phase II clinical trial in adults suffering from acute non-lymphoblastic leukemia, the dose-limiting toxicity was mucositis, which resulted from continuous infusion of alanosine at a dose of about 125 mg/m² body weight for 5 days.

Notwithstanding previously reported toxicities stemming from alanosine administration, MTAP-deficient cancers respond to far smaller doses of alanosine. Moreover, the cells' susceptibility to adenine starvation and lack of MDR renders them sensitive to treatment with alanosine, alone or conjunction with other chemotherapeutic agents.

As described above, particularly preferred uses of the compositions of the invention are in the treatment of diseases and disorders wherein the cells responsible for the disease or disorder are MTAP-deficient. Whether a patient has a disease characterized by cells that are MTAP deficient can be determined using any suitable assay. Representative examples include nucleic acid amplification-based assays to assess whether the cancer cells lack the gene encoding MTAP (see, e.g., U.S. Pat. No. 6,214,571), as well as immunohistochemical and biochemical assays for MTAP enzymatic activity.

4. Administration

The compounds of this invention are administered in a therapeutically effective amount to a subject in need of treatment. Administration of the compositions of the invention can be via any of suitable route of administration, particularly parenterally, for example, intravenously, intra-arterially, intraperitoneally, intrathecally, intraventricularly, intraurethrally, intrasternally, intracranially, intramuscularly, or subcutaneously. Such administration may be as a single bolus injection, multiple injections, or as a short- or long-duration infusion. Implantable devices (e.g., implantable infusion pumps) may also be employed for the periodic parenteral delivery over time of equivalent or varying dosages of an alanosine formulation according to the invention. See, e.g., U.S. Pat. Nos. 6,743,204; 6,723,039; 6,694,191; 6,036,459; 5,840,069; and 4,692,147. For such parenteral administration the compounds are preferably formulated as a sterile solution in water or another suitable solvent or mixture of solvents. The solution may contain other substances such as salts, sugars (particularly glucose or mannitol), to make the solution isotonic with blood, buffering agents such as acetic, citric, and/or phosphoric acids and their sodium salts, and preservatives. The preparation of suitable, and preferably sterile, parenteral formulations is described in detail in the section entitled “Compositions”, above.

In the context of this invention, actual alanosine dosage levels in the compositions of this invention can be varied so as to obtain an amount of the active compound(s) that is effective to achieve the desired therapeutic response for a particular patient, compositions and mode of administration. In general, daily administration or continuous infusion of L-alanosine at dosages less than those known to produce toxicities will be the preferred therapeutic protocol to enhance the anti-metabolite activity of the drug. The selected dosage level will depend upon the activity of the particular compound, the route of administration, the severity of the condition being treated and the condition and prior medical history of the patient being treated. However, it is within the skill of the art to start doses of the compound at levels lower than required to achieve the desired therapeutic effect and to gradually increase the dosage until the desired effect is achieved.

With regard to human and veterinary treatment, the amount of alanosine administered will, of course, be dependent on a variety of factors, including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; the judgment of the prescribing physician or veterinarian; and like factors well known in the medical and veterinary arts.

Notwithstanding the foregoing, therapeutically effective amounts of L-alanosine for treatment of mammals preferably range from about 50 mg/m² to about 4 g/m², most often about 80 mg/m² to about 125 mg/m² or a dosage sufficient to achieve about 1000-2000 nM concentration in blood within 24 hours of administration. For treatment of MTAP-deficient cells, dosages at the lower end of the dosage range are preferred. In those cases where L-alanosine is administered in conjunction with another chemotherapeutic agent, it is preferred to take into account the total drug burden being placed on the patient. If desired, the effective daily dose can be divided into multiple doses for purposes of administration; consequently, single dose compositions may contain such amounts or submultiples thereof to make up the daily dose.

In a preferred embodiment, a patient receives L-alanosine at a dosage of 80 mg/m² daily, administered CIVI on a 21-day cycle using an ambulatory infusion pump. A patient's body surface area (BSA) can be calculated using the formula:
m²=[height(cm)×weight(kg)/3600]^{0.5} (II)

For example, if a patient is 175 cm tall and weighs 80 kg, the BSA is 1.96 m². The starting dose for the patient would be 157 mg/day, and the total dose over five days will be 784 mg infused from a 250 ml bag of 0.9% normal saline. As IV infusion bags frequently contain a 10% overfill (total volume=275 mL), the total amount of drug needed would be: 784 mg×(275/250)=862 mg. This amount of drug can be prepared using 43.1 mL of a 20 mg/mL stock solution of reconstituted L-alanosine (or L-alanosine prepared from a stable liquid formulation). To prepare the infusion solution, 43.1 ml of saline is first removed from the IV bag containing 275 mL, after which 43.1 mL of the 20 mg/mL stock solution of L-alanosine is added. The infusion pump is then programmed to deliver 250 mL over the five day period, corresponding to a delivery rate of approximately 2.1 mL/hr.

Over the course of therapy, it may be desired to increase the dosage of L-alanosine, or, in the event of a drug toxicity (e.g., stomatitis/mucositis), to decrease the L-alanosine dosage. For example, in patients who do not experience hematologic or non-hematologic toxicities after at least one cycle of L-alanosine therapy at a starting dose of 80 mg/m²/day, dosages can be increased from 10 to 50% or more. A preferred dosage escalation is a 25% dosage increase over the previous dosage. If, as a result of increasing dosage, a patient develops a hematologic or non-hematologic toxicity, the supervising practitioner may elect to continue or discontinue the infusion, depending on the degree of toxicity. If the infusion is discontinued, a return to the next lowest dosage level should be implemented during the next cycle. Thus, if toxicity develops during the initial stage of alanosine treatment (for example, during the course of the five day infusion), the supervising practitioner may elect to continue or discontinue the infusion, depending on the degree of toxicity. In the event the infusion is discontinued, the following round of treatment is preferably at a reduced dosage.

### EXAMPLES

The following Examples are provided to illustrate certain aspects of the present invention and to aid those of skill in the art in practicing the invention. These Examples are in no way to be considered to limit the scope of the invention in any manner.

### Example 1

### Stable Liquid Alanosine Formulations

This example describes experiments relating to stable liquid formulations of L-alanosine. Previous reported formulations were lyophilized compositions containing L-alanosine, monosodium, at a pH around 7 that required reconstitution at or around the time of administration using a suitable diluent having a pH at or near a physiological pH.

1. Titration

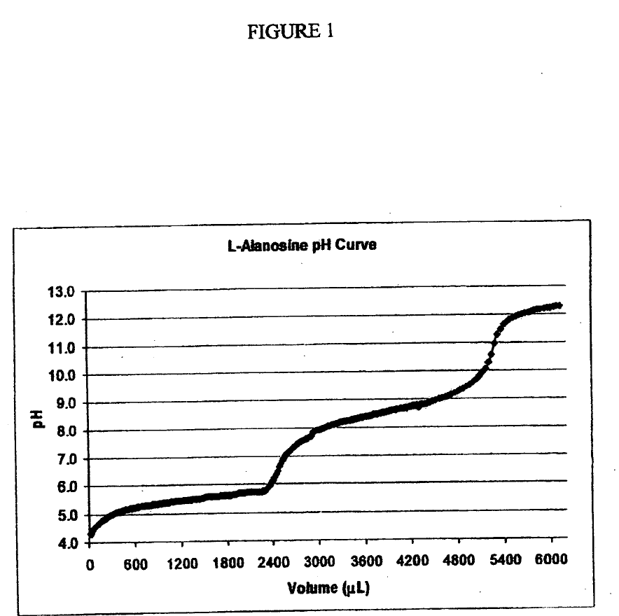

Initially, a titration curve was generated by adding L-alanosine (free base) in water to a concentration of approximately 20 mg/mL. The solution was stirred on a stir plate at room temperature and the pH was adjusted by adding 20 microliter (μL) aliquots of 1N NaOH. After the addition of each aliquot, the resulting pH was measured potentiometrically and the results were recorded. The results of the titration appear in FIG. 1. The solution was also visually inspected during the course of the titration experiment to observe full dissolution of the alanosine. As expected, the free base material exhibited poor solubility, and it did not fully dissolve until the pH exceeded 6.2. As seen in FIG. 1, that portion of titration curve between pH 4.3 and 5.9, after the initial buffering region and before the equivalence point, is unusual. Without wishing to be bound to a particular theory, this is likely due to the compound's poor solubility in water because the material does not fully dissociate to form the monosodium salt until there are sufficient free hydroxide ions (from the NaOH) in solution. The curve also has two inflection points, indicating that there are two buffering regions and two pKa's at approximately 5.5 and 8.5.

2. Temperature and pH Effects on Stability

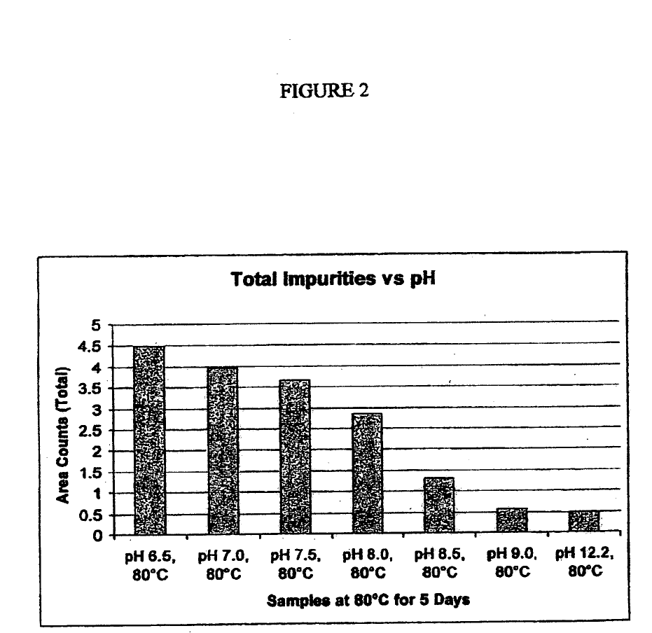

Stability studies were then performed at different pHs. To do this, a solution of about 20 mg/mL L-alanosine in water, pH 6.5 was prepared. The pH of the solution was then adjusted in increments of 0.5 using 1N NaOH. Stability measurements of two duplicate samples were taken at each increment, beginning at pH 6.5 up through pH 9.0, and one sample was taken at pH 12.2. One set of samples was stored at 5° C., the other at 80° C., for five days, after which they were analyzed for pH, physical appearance, osmolality, and impurities. FIG. 2 shows the relative purity of the samples stored at 80° C. for five days as measured by HPLC. These results show an inverse relationship between the amount of impurities and pH, with the lowest levels of impurities being found in samples having a pH of 9.0 or 12.2. Here, impurities were determined by reverse phase HPLC as described herein. The physical appearance of the solutions also showed a pH dependence at the elevated temperature. The solutions were different shades of yellow, beginning with a darker yellow at the lowest pH and becoming increasing lighter with increasing pH. Together, the results with regard to purity and color imply that L-alanosine degrades at lower pH and that the degradation products have a greater extinction coefficient than L-alanosine in the yellow region.

The osmolality of samples having a pH of 6.5 were 225 and 247 milliosmols (mOsm) when stored at 5° C. and 80° C., respectively, whereas the osmolality of samples having a pH of 8.0 were 272 and 273 mOsm when stored at 5° C. and 80° C., respectively. The solution is isotonic and, if desired, excipients may be added to increase the osmolality. It was also observed that the pH of samples stored at 80° C. for 5 days shifted toward pH 8-8.5.

3. Long-Term Stability of Liquid Formulation

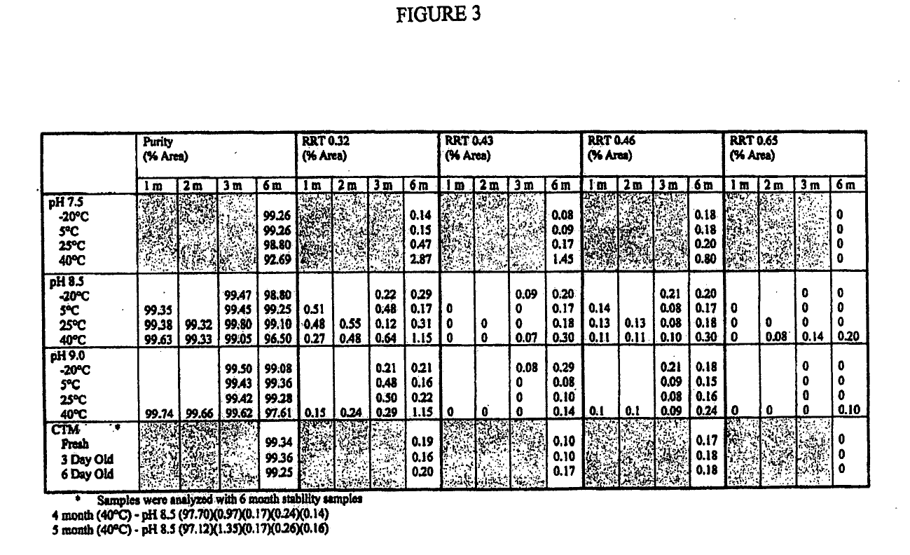

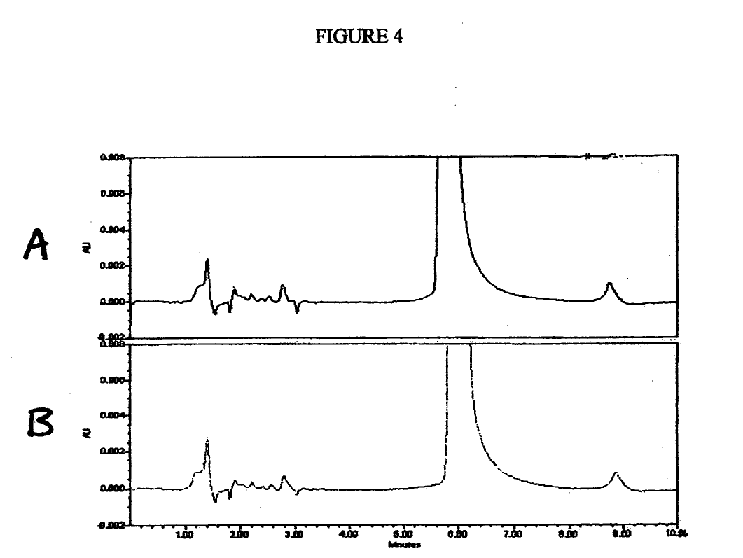

The long-term stability of L-alanosine formulations in a basic solution was studied at different pHs. Batches of 20 mg/mL L-alanosine in water at different pHs, i.e., 7.5, 8.5, and 9.0 (adjusted using 5N NaOH), were prepared. Each solution was divided into 5 mL aliquots and placed in 6 mL vials sealed with Teflon-faced screw caps (VWR Scientific, catalogue No. 66011-880). Vials of each solution were placed at five different temperatures, −20° C., 5° C., 25° C., 40° C., and 60° C., and aliquots were analyzed at various time points to determine stability. For comparison, an aliquot of freshly reconstituted L-alanosine (clinical trial lyophilizate dissolved in 5 mL H₂O by shaking) was also assayed along with the liquid formulation samples. The results of this time course experiment are shown in FIGS. 3 and 4. As shown in FIG. 3, the stability of L-alanosine formulated at different pHs is similar to the results described in part 2 of this Example. FIG. 4 confirms that an aqueous formulation containing L-alanosine and having a pH of 8.5 is as stable after storage at 5° C. for six months as a composition prepared by freshly reconstituting lyophilized L-alanosine.

To assess the conversion of L-alanosine to D-alanosine over time under different storage conditions, several solutions of L-alanosine having different basic pHs (i.e., 7.5 and 8.5) were prepared, as described above. Again, each solution was divided into 5 mL aliquots and placed in 6 mL vials sealed with Teflon-faced screw caps. Vials of each solution were placed at either 5° C. or 25° C., and aliquots were analyzed at various time points to determine stability. Initially, to determine the relative HPLC elution profiles of a D- and L-alanosine, three solutions containing approximately 50/50 (w/w) compositions of D- and L-alanosine were also prepared, as above, and subjected to reverse HPLC to determine the retention times and concentrations of the two stereoisomers. The results of these experiments appear in Table 1, below.

TABLE 1
Chirality Analysis

| | | | Retention | | % Total | |
|---|---|---|---|---|---|---|
| Compo- | | | Time (min.) | | Peak Area | |
| sition | pH | Temp. | L | D | L | D |
| D/L - t₀ | N/A | N/A | 16.941 | 17.375 | 50.12 | 49.88 |
| D/L - t₀ | N/A | N/A | 17.083 | 28.657 | 50.10 | 49.90 |
| D/L - t₀ | N/A | N/A | 17.375 | 29.155 | 50.46 | 49.54 |
| L - t₀ | N/A | N/A | 18.003 | no peak | 100 | N/A |
| L - t₀ | N/A | N/A | 18.341 | no peak | 100 | N/A |
| L - t₀ | N/A | N/A | 18.695 | no peak | 100 | N/A |
| L - t_{6 mo.} | 8.5 | 5° C. | 21.300 | no peak | 100 | N/A |
| L - t_{6 mo.} | 8.5 | 5° C. | 21.607 | no peak | 100 | N/A |
| L - t_{6 mo.} | 8.5 | 25° C. | 21.817 | 36.533 | 97.97 | 2.03 |
| L - t_{6 mo.} | 8.5 | 25° C. | 22.080 | 36.883 | 97.96 | 2.04 |
| L - t_{1 yr.} | 7.5 | 5° C. | 20.300 | no peak | 100 | N/A |
| L - t_{1 yr.} | 7.5 | 5° C. | 20.525 | no peak | 100 | N/A |
| L - t_{1 yr.} | 7.5 | 25° C. | 20.735 | 34.800 | 99.01 | 0.99 |
| L - t_{1 yr.} | 7.5 | 25° C. | 20.995 | 35.117 | 99.22 | 0.78 |
| L - t_{1 yr.} | 8.5 | 5° C. | 19.031 | no peak | 100 | N/A |
| L - t_{1 yr.} | 8.5 | 5° C. | 19.409 | no peak | 100 | N/A |
| L - t_{1 yr.} | 8.5 | 25° C. | 19.690 | 33.115 | 95.55 | 4.45 |
| L - t_{1 yr.} | 8.5 | 25° C. | 19.977 | 33.560 | 95.44 | 4.56 |

These results show that after one year, liquid samples of L-alanosine stored at 5° C. showed no conversion to D-alanosine regardless of whether the solution had a pH of 7.5 or 8.5; however, storage at an elevated temperature (here, 25° C.) resulted in the conversion of a small percentage of L-alanosine to D-alanosine at either pH of 7.5 or 8.5, although about 5-10 times as much L-alanosine was converted to D-alanosine when the solution had a pH of 8.5 as compared to when the solution had a pH of 7.5. Thus, these results indicate that the higher the pH of formulations stored at elevated temperatures (e.g., 25° C.), the higher the D-alanosine content over time.

The following HPLC procedure was used to separate the L- and D-enantiomers of alanosine. The HPLC system (Agilent 1100) employed a 4.6 mm×150 mm column (Phenomenex) containing 5 μm particles (Chirex 3126, (D)-penicillamine) as the stationary phase. The system was equipped with both UV and polarimeter detectors. The UV detector was set to 254 nm to monitor products being eluted from the column, while the polarimeter detector was set to 670 nm. The eluent was 2 mM CuSO₄/MeOH 70-30. The flow rate was 1.7 mL/min. at a pressure of 200 bar. For each run, a 100 μL sample containing 0.5 mg/mL in 2 mM CuSO₄ was injected onto the column. In each case, L-alanosine eluted first, after about 20 minutes, with the D-enantiomer eluting at around 34 minutes. To reverse the elution order, (L)-penicillamine could be used.

4. Arrhenius Evaluation

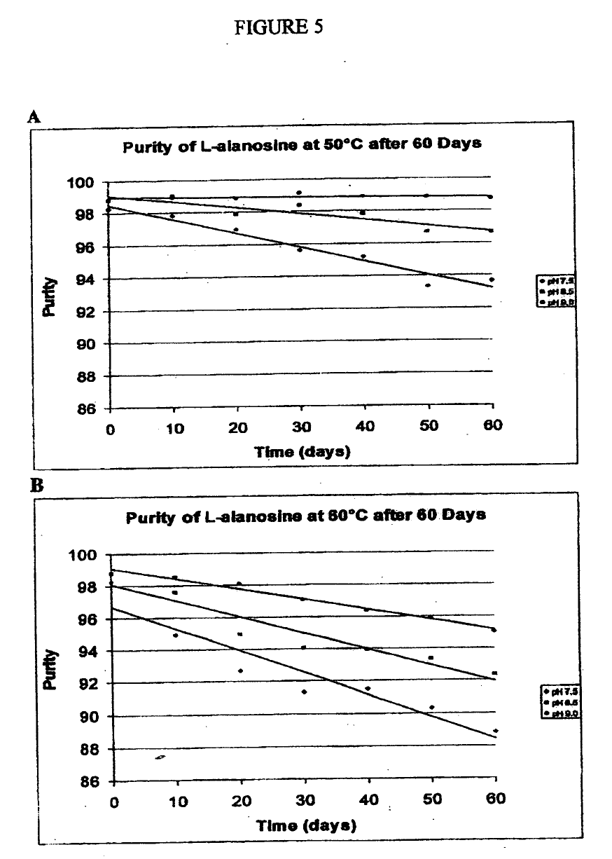

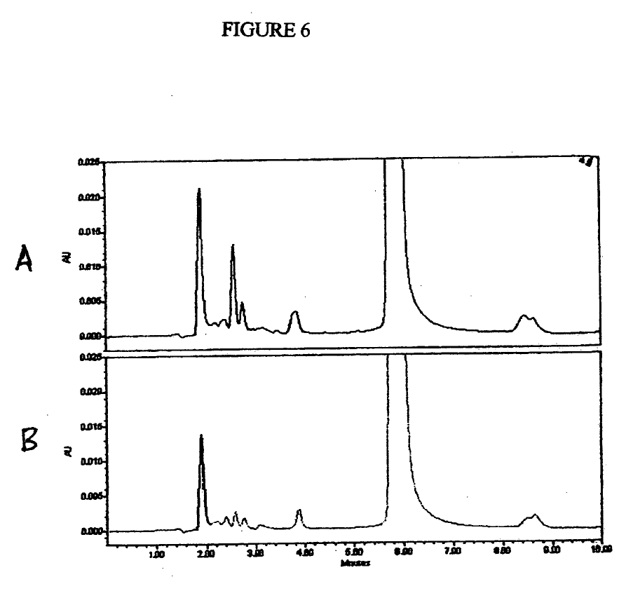

An Arrhenius analysis was performed to predict the loss of potency and increase in degradation products over time for commonly used storage temperatures for many drugs. In this experiment, solutions of 20 mg/mL of L-alanosine at pHs 7.5, 8.5, and 9.0 were prepared at room temperature and divided into 5 mL samples aliquotted into 6 mL vials. Sets of the different solutions were then placed at 40° C., 50° C., or 60° C., and samples were taken every ten days over a 60 day period. The results of this study for the 50° C. and 60° C. storage conditions are shown in FIG. 5. Samples taken from the solutions stored at 40° C. showed no trend in decreased purity of alanosine. As shown in FIG. 5, samples taken at different times from the solutions stored at 50° C. or 60° C. showed trends in decreasing purity over the time course of the study. Specifically, these plots show a distinct difference in purity as a function of pH and temperature. FIG. 6 shows two HPLC chromatograms comparing samples of the pH 7.5 and 8.5 liquid formulations stored at 50° C. for 60 days (chromatograms A and B, respectively). These plots show that greater levels of impurities are formed in the pH 7.5 formulation over time as compared to the pH 8.5 formulation. As seen from these chromatograms, four major impurities were detected, with relative retention times (RRTs) of 0.32, 0.43, 0.46, and 0.63, respectively. In the pH 7.5 formulation, there was more of each of these four impurities as compared to the pH 8.5 formulation. The impurity at RRT 0.32 formed readily as a function of temperature and pH. The impurities at RRTs 0.43 and 0.46 also formed as a function of temperature and pH, but not as readily. The impurity at RRT 0.65 was strictly a high temperature degradant (at 50° C. and 60° C.), as it did not form at 40° C.

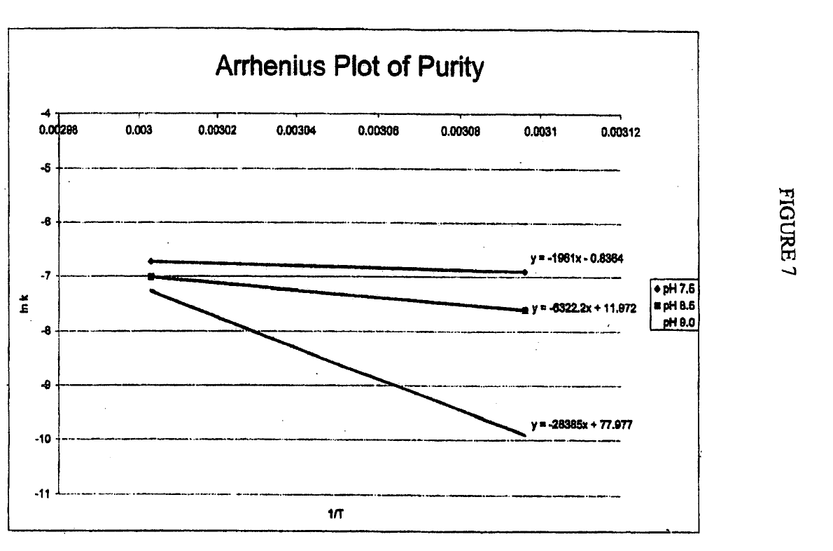

From these data, it is apparent that the purity of L-alanosine in solution decreases over time at pH 7.5, while there is much less degradation at pH 8.5 and pH 9.0. Indeed, the purity of the samples is similar at pH 8.5 and 9.0. These results confirm that L-alanosine is unstable in solution over time at lower pH (i.e., below about pH 8) as compared to higher pH (i.e., pH 8 and above). The trends in the data shown in FIG. 5 allow an Arrhenius analysis to be performed to predict changes in purity over time at various temperatures, particularly 5° C. and 25° C. Linear regression was used to determine the positions of the lines shown in FIG. 5, which then allowed the slope of the lines (i.e., the reaction rates, per day) to be determined for each pH and a given temperature. Arrhenius plots were the generated for each of pHs 7.5, 8.5 and 9.0 from the reaction rate versus the inverse temperatures. These plots are shown in FIG. 7, which allow the equation of the lines for each of three liquid formulations to be calculated. From these equations, the purity of any given temperature for a particular formulation at a given time can be predicted.

From the Arrenhius analysis, the amount of degradation or loss of purity over time, e.g., a two-year period, was determined for the different pH values and temperatures as shown in Table 2, below.

TABLE 2
Degradation and Purity

| pH | | Amount degraded/day (%) | Predicted purity after 2 years |
|---|---|---|---|
| 7.5 | 5° C. | 0.000374 | 99.727% |
| | 25° C. | 0.000601 | 99.561% |
| 8.5 | 5° C. | 2.1 × 10⁻⁵ | 99.985% |
| | 25° C. | 9.66 × 10⁻⁵ | 99.930% |
| 9.0 | 5° C. | 3.34 × 10⁻¹¹ | 100.00% |
| | 25° C. | 3.12 × 10⁻⁸ | 100.00% |

These results indicate that aqueous formulations containing 20 mg/mL L-alanosine and having a pH of between 8.5-9.0 are sufficiently stable to be used as human pharmaceutical preparations for a period of at least two years. Moreover, while it may be preferred to refrigerate the compositions while in storage, it is not necessary to do so, as calculations indicate that the L-alanosine will remain more than 99.9% pure when stored at 25° C.

5. HPLC Analyses

The following High Performance Liquid Chromatography (HPLC) method was used for identifying impurities in compositions containing L-alanosine, as described in part (4) of this example. The HPLC system (Waters Corp., model 2690 Gradient HLPC System) employed a 4.6 mm×250 mm column (MetaChem Inc.) containing 5 μm particles (Inertsil ODS-3) as the stationary phase. The system was equipped with a UV/VIS detector (Waters Corp., model 996) set to 226 nm to monitor products being eluted from the column. Data generated from the column was managed on a personal computer running Empower Software (Waters Corp.). After preparation, the column was washed with at least 100 mL of water/acetonitrile (90:10, v/v), and stored at ambient temperature in the same solution.

In each HPLC run, the mobile phase, flowing at 1.0 mL/min., comprised 7.0 g of anhydrous monobasic potassium phosphate (KH₂PO₄; JT Baker) dissolved in 1000 mL H₂O (at least HPLC grade). The pH of the solution was adjusted to 2.5 using phosphoric acid (85%, Fisher Scientific). 2.4 g of 1-decane sulfonic acid, sodium salt (Avocado Research Chemicals) was then mixed into the solution. 50 mL of acetonitrile (HPLC Grade, Burdick & Jackson) was then mixed with 950 mL of this solution. This mixture was then degassed by filtering through Durapore membrane filters, 0.45 μm, Type HVLP, under vacuum. The column flow rate was set at 1.0 mL/min.

To prepare an alanosine standard solution, 5 mg of L-alanosine was dissolved in 25 mL of diluent in a 25 mL volumetric flask. Diluent was prepared by dissolving 3.5 g KH₂PO₄ and 3.5 g (Na₂HPO₄, Mallincrodt) in 1000 mL of HPLC grade H₂O. The final concentration of L-alanosine was calculated from the actual weight of the standard solution (ca. 0.2 mg/mL). Once prepared, an alanosine standard solution was stored at ambient temperature and retained for no more than seven days.

In addition to an alanosine standard solution, a sensitivity standard was also prepared by diluting 1 mL of the alanosine standard solution 1:100 using diluent. 1 mL of this intermediate solution was then placed in a 10 mL volumetric flask and diluted and mixed well with diluent to produce a solution containing 0.2 μg/mL L-alanosine.

HPLC runs were performed after the column had equilibrated for at least three hours in the mobile phase at room temperature. Column suitability was assessed at the beginning and end of each series of experiment as follows. Initially, two 10 μL aliquots of diluent were injected onto the column in duplicate, and the chromatographs recorded and examined. After confirming the absence of interference in the region of interest (L-alanosine elutes after approximately 5.9 min.), six 10 μL aliquots of the alanosine sensitivity standard were injected onto the column. Next, three 10 μL aliquots of the L-alanosine standard solution were injected onto the column, followed by 10 μL aliquots of the test compositions to be assayed. If more than ten test samples were to be tested, after every tenth test sample, a single 10 μL aliquot of the L-alanosine standard solution was injected. The suitability of the column was confirmed when the RSDs of the peak areas for all injections of the L-alanosine standard were less than 2.0% with an average tailing factor of less than 1.8, and the RSDs of the peak areas for the six alanosine sensitivity standard aliquots were less than 15.0%.

6. Long-Term Stability of Large-Scale Production Lot

The stability of a lot of L-alanosine produced from a 25 L batch culture of S. alanosinicus culture, as described in U.S. Pat. No. 3,676,490. A total of 1,250 vials of a stable liquid formulation of L-alanosine resulted from this production run (for each vial, 5 mL of the stable liquid L-alanosine formulation aliquotted into a 20 mL type 1 Schott vial then sealed with a gray bromobutyl stopper). Shelf-life stability of the lot was assessed over a six-month period by storing vials at either 5° C.±3° C. or 25° C.±2° C. in either an inverted or upright position. For vials stored in an inverted position, samples were withdrawn for analysis at 0, 1, 3, and 6 month time points, while for those stored in an upright position, samples were only taken at the 3 and 6 month time points. Sampling and stability testing were performed as described above. In each instance the sample tested met or exceeded the threshold specification for the particular parameter being assessed. These parameters (specification in parentheses) were: solution color (clear, colorless to yellow); L-alanosine activity (90.0-110.0% of label claim); related substance eluted at 0.47 RTT (less than 2%); total related substances (less than 5%); and pH value (8.0-9.0). These results further confirm the stability of the instant liquid L-alanosine formulations.

### Example 2

### Drug Combinations

This example describes cell-based experiments in which the effects of L-alanosine in combination with each of six other chemotherapeutic agents, docetaxel (Taxotere®), 5-fluorouracil (5-FU), vinorelbine, pemetrexed (Alimta®), gefitinib (Iressa®), and gemcitabine, were tested. In each experiment, tumor cells from lung, pancreatic, or mesothelial tumor cell lines were plated in 96-well plates at 5,000 cells/well in 100 μL media. The media for all assays except the Alimta® studies was RPMI-1640 and 10% horse serum. For the Alimta® studies, the media used was RPMI 1640 with dialyzed fetal bovine serum. All media contained 100 Units/mL penicillin and 100 μg/mL streptomycin. The cells were allowed to adhere for 18 hours at 37° C./5.0% CO₂. Titrated concentrations of Taxotere® (docetaxel), 5-FU, vinorelbine, Iressa®, Alimta®, or gemcitabine, alone or with titrated concentrations of L-alanosine, were added to the culture medium in each well. Drugs were dosed at the ratios listed in Table 3, below. After drug addition, cells were incubated three days at 37° C., 5.0% CO₂. Cell viability was assayed using a standard MTT assay, as follows: 10 μl of 12 mM 3-[4,5-dimethylthiazol-2-yl]-2,5-diphenyltetrazolium bromide (MTT) (Sigma) were added to each well. The cells were incubated at 37° C., 5% CO₂ for 4 to 6 hours. 100 μl of 20% SDS, 0.015 M HCl were added to each well and the cells were incubated overnight. Cytotoxicity was measured by reading absorbance at 595 nM. The results are summarized in Table 3, below.

TABLE 3
Effects of Drug Combinations with L-alanosine

| Cell line (ratio | | | EC90 CI | | EC75 CI | |
|---|---|---|---|---|---|---|
| drug:alanosine | Cancer Type | Drug | value | Effect | value | Effect |
| A549 (1:100) | NSCLC | Taxotere ® | 0.7 | moderate | 0.8 | moderate |
| | | | | synergism | | synergism |
| NCI-H2322M | NSCLC | Taxotere ® | 0.6 | synergism | 0.9 | light |
| (1:1000) | | | | | | synergism |
| NCI-H292 | NSCLC | Taxotere ® | 0.9 | additive | 1.1 | additive |
| (1:1000) | | | | | | |
| PanC1 (1:1000) | pancreatic | Taxotere ® | 0.6 | synergism | 0.6 | synergism |
| PanC1 (1:1) | pancreatic | 5-FU | 0.4 | synergism | 0.6 | synergism |
| BX-PC3 (5:1) | pancreatic | 5-FU | 0.8 | moderate | 0.8 | moderate |
| | | | | synergism | | synergism |
| BX-PC3 | pancreas | vinorelbine | 0.6 | synergism | 0.6 | synergism |
| (1:100) | | | | | | |
| BX-PC3 (1:4) | pancreas | Iressa ® | 0.28 | strong | 0.29 | strong |
| | | | | synergism | | synergism |
| NCI-H2452 | mesothelioma | 5-FU | 0.5 | synergism | 0.5 | synergism |
| (1:1) | | | | | | |
| NCI-H2452 | mesothelioma | Taxotere ® | 0.6 | synergism | 0.8 | moderate |
| (1:1000) | | | | | | synergism |
| NCI-H2452 | mesothelioma | gemcitabine | 0.6 | synergism | 0.6 | synergism |
| (1:100) | | | | | | |
| NCI-H2452 | mesothelioma | Alimta ® | 0.3 | strong | 0.3 | strong |
| (1:1) | | | | synergism | | synergism |
| A549 (1:10) | NSCLC | Alimta ® | 0.9 | Slight | 0.9 | slight |
| | | | | synergism | | synergism |

Data analysis was performed using GraphPad Prism version 3.0 (GraphPad Software, San Diego, Calif. USA). Taxotere®, 5-FU, vinorelbine, and gemcitabine each showed additive or synergistic effects with L-alanosine in terms of combinatorial index measurements in all assays except one, as determined using the Calcusyn Windows Software for Dose Effect Analysis (Biosoft, Ferguson, Mo., USA), while L-alanosine in combination with Alimta® showed strong synergism in the context of mesothelioma cells, but only slight synergism with non-small cell lung cancer cells. In addition, L-alanosine in combination with Iressa® showed strong synergism in the context of pancreatic cancer cells. The combinatorial index equation used to calculate the results above was based on the multiple drug-effect equation of Chou-Talalay derived from enzyme kinetic models (Chou and Talalay (1977), J. Biol. Chem., vol. 252:6438-6441; Chou and Talalay (1983), Trends Pharmacol. Sci., vol. 4:450-454).

### Example 3

### Effects of L-Alanosine in Combination with Pemetrexed

This example describes cellular analyses in which effects of L-alanosine in combination with pemetrexed (Alimta®) were tested. Pemetrexed (Alimta®) is a multi-targeted antifolate that acts on a number of folate-dependent enzymes, including thymidylate synthase, dihydrofolate reductase, glycinamide ribonucleotide formyltransferase (GARFT), and aminoimidazole carboxamide ribonucleotide formyltransferase (AICARFT). In the analyses, the ATP-lowering activity of pemetrexed was determined in cell lines from non-small cell lung cancer (NSCLC), mesothelioma, and pancreatic cancer; the effects of engaging the MTAP pathway on pemetrexed activity was determined; and the anti-tumor efficacy of the combination of L-alanosine (sometimes referred to hereafter as “SDX-102”) and pemetrexed was determined in MTAP-negative tumor cells.

Pemetrexed lowered intracellular ATP levels in several cell lines. Treatment with pemetrexed caused a 50% reduction of intracellular ATP after 72 hours incubation at concentrations ranging from 80 nM (NCI-H2452) to 5 μM (BXPC3). MTAP status was determined using immunoblot analyses and correlated with the ability of an MTA analog to rescue ATP levels in cell lines treated with SDX-102. ATP levels were measured using Cell-Titer Glow™ (Promega) after a 3 day treatment with titrated concentrations of either SDX-102 or pemetrexed. Cell viability was assessed by an MTT assay measured after 3 days after treatment with L-alanosine or pemetrexed. Results are shown in Table 4, which shows activities of SDX-102 and pemetrexed in MTAP positive and negative pancreatic cancer, NSCLC, and mesothelioma cell lines.

TABLE 4
Effects of L-alanosine and Permetrexed on Tumor Cell Lines

| | | | SDX-102 | SDX-102 | Alimta ® | Alimta ® |
|---|---|---|---|---|---|---|
| | | | ATP | MTT | ATP | MTT |
| | | MTAP | IC₅₀ | IC₅₀ | IC₅₀ | IC₅₀ |
| Cell Line | Tumor Type | status | (μM) | (μM) | (μM) | (μM) |
| BxPC3 | Pancreatic | Negative | 0.6 | 6 | 5 | 0.05 |
| HS-766T | Pancreatic | Positive | 2 | >20 | 0.8 | >20 |
| A-594 | NSCLC | Negative | 0.3 | 0.4 | >20 | 0.5 |
| A-427 | NSCLC | Positive | 1.5 | 3.3 | 5 | 3 |
| NCI-H2452 | Mesothelioma | Negative | 0.4 | 0.5 | 0.1 | 0.05 |
| NCI-H226 | Mesothelioma | Positive | 0.4 | 1.5 | 1 | 1 |

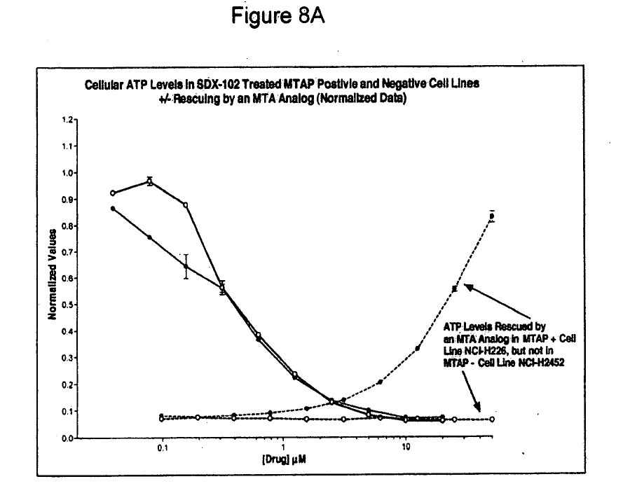

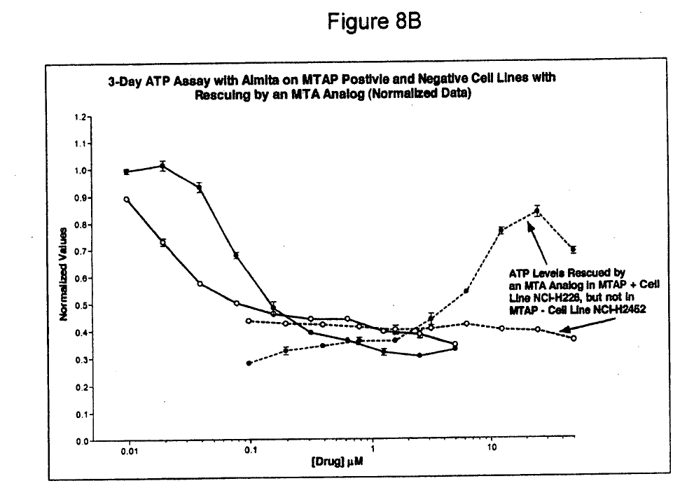

Activation of the purine salvage pathway, using an MTAP-substrate, was sufficient to block the pemetrexed-induced ATP depletion in the MTAP-expressing cells, but not in the MTAP-deleted cells. In MTAP-positive cells (HS-766T, A-427, and NCI-H226) treated with pemetrexed, ATP levels were restored to more than 85% of the level measured in control cells by addition of an MTAP substrate. The MTAP substrate was able to fully rescue HS-766T cells from loss of viability induced by pemetrexed as measured by the MTT assay. ATP values measured using Cell-Titer Glow™ (Promega) after 3 days exposure to the MTA analog. Solid lines in FIG. 8 represent the effect of titrations of SDX-102 from 0.05 μM-20 μM (FIG. 8A) or pemetrexed from 0.01 μM-5 μM (FIG. 8B) on cellular ATP levels. The dotted lines represent the effect of a single concentration of SDX-102 (10 μM) or pemetrexed (2.5 μM) with a titration of an MTA analog from 0.1 μM-50 μM. Treatment with the MTA analog rescues ATP levels in the MTAP positive cell line NCI-H226 but not in the MTAP negative cell line NCI-H2452 following treatment with either SDX-102 and pemetrexed.

Low concentrations of SDX-102 enhanced the cytotoxic activity of pemetrexed in several MTAP-deleted cell lines. In the mesothelioma cell line NCI-H2452, incubation with 200 nM L-alanosine shifted the IC₇₅ of pemetrexed from 200 nM to 15 nM. SDX-102 in combination with pemetrexed displayed strong synergism in a mesothelioma cell line and a more modest synergistic effect in a NSCLC cell line (A-549). The indicated cell lines were exposed to pemtrexed and SDX-102 alone or in combination at the ratios indicated in Table 5.

TABLE 5
Effects of L-alanosine and Pemetrexed

| | | Ratio | EC₇₅ CI | |
|---|---|---|---|---|
| Cell Line | Tumor Type | (Alimta:SDX-102) | Value | Effect |
| NCI-H2452 | Mesenthelioma | 1:1 | 0.3 | strong |
| | | | | synergism |
| A-549 | NSCLC | 1:10 | 0.9 | synergism |

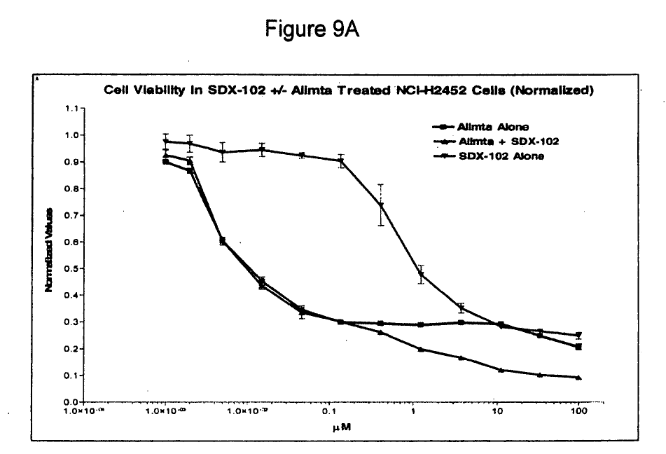

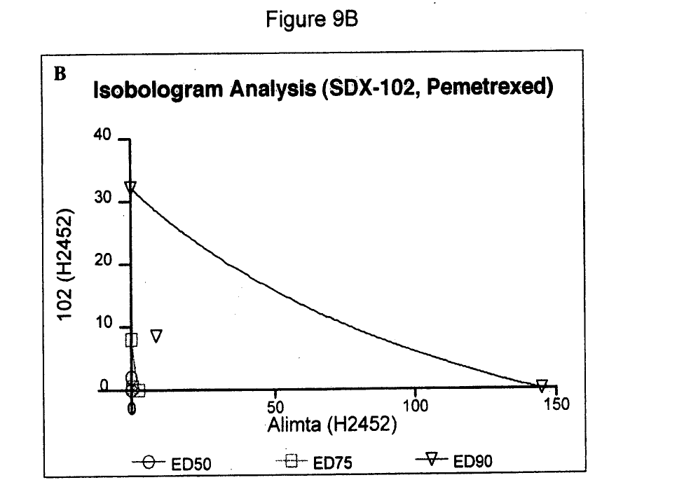

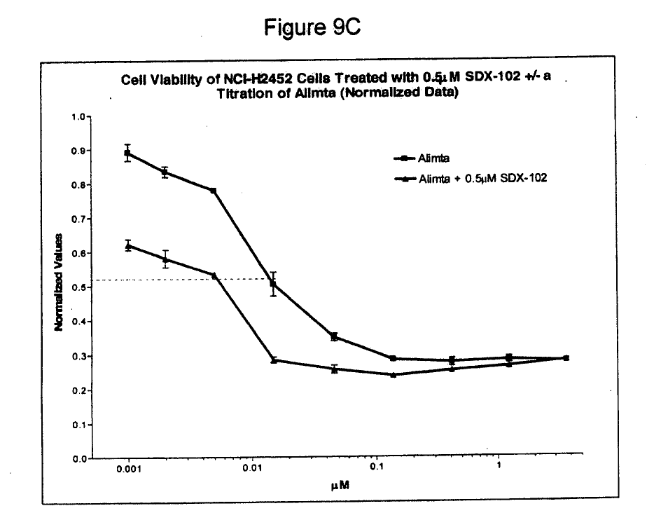

After 72 hours of treatment, cytotoxicity curves were generated using an MTS assay. Drug combination effects were defined using the Combinatory Index Method as described by Chou and Talalay as calculated by the CalcuSyn Software for Dose Effect Analysis (Biosoft, Cambridge, U.K.). FIGS. 9A-9C show results from these analyses for the mesothelioma cell line NIC-H2452. FIG. 9A shows a viability assay in NCI-H2452 mesothelioma cells treated with SDX-102 in combination with pemetrexed for 72 hours. Drugs were dosed from 1 nM-100 μM, alone or in combination. These results demonstrate an increased cytotoxic effect at IC₇₅ levels when SDX-102 and pemetrexed are combined. FIG. 9B shows an isobologram analysis of the curves from FIG. 9A using CalcuSyn Software for Dose Effect Analysis (Biosoft, Cambridge, U.K.). The “hyperbolic” lines represent dose combinations of SDX-102 and pemtrexed that should produce “additive” effects. Any points above the curves are considered antagonistic while any points below the curve indicate synergy. The points represent experimental EC₅₀, EC₇₅, and EC₉₀ values for the combination of the two drugs. The analysis indicates synergy when combining SDX-102 and pemetrexed. FIG. 9C shows a viability assay (MTS) performed with a constant concentration of SDX-102 (0.5 μM) and a titration of pemetrexed from 1 nM-5 μM. This graph shows an increased cytotoxic effect when adding pemetrexed to a constant concentration of SDX-102.

In conclusion, pemetrexed (Alimta®) treatment lowered intracellular ATP pools in NSCLC, pancreatic cancer, and mesothelioma cell lines. When MTAP-expressing cell lines are supplemented with a methylthioadenosine (MTA) analog, the ATP pool depletion caused by exposure to pemetrexed was rescued. This effect was not observed in MTAP-negative tumors. Thus, the ATP reduction caused by pemetrexed was due to inhibition of the de novo purine synthesis pathway. Tumors that possess an intact purine salvage pathway (MTAP-expressing) should be less sensitive to the anti-tumor activity of pemetrexed, and tumors that express no MTAP should be more sensitive to pemetrexed. Also, it was determined that L-alanosine was a selective inhibitor of the de novo purine pathway that displayed enhanced cytotoxicity in MTAP-negative cells with an inactive purine salvage pathway. It was determined that SDX-102 and pemetrexed displayed a highly synergistic combination effect in MTAP-negative mesothelioma cells. A more modest synergistic effect and additivity were observed in MTAP-negative NSCLC and pancreatic cancer cell lines treated with L-alanosine and pemetrexed.

### Example 4

### Effects of L-Alanosine in Combination with Docetaxel or 5-FU

This example describes cell-based and anaimal-based analyses in which effects of L-alanosine in combination with Docetaxel (Taxotere®) or 5-Fluorouracil were tested. Docetaxel is a microtubule-stabilizer anti-neoplastic agent, is structurally related to paclitaxel, and is widely used in several indications, including non-small cell lung cancer (NSCLC). Fluorouracil (5-FU) is an anti-metabolite also frequently used in several cancer indications, including pancreatic cancer and NSCLC. This analyses compared the activity of docetaxel and 5-FU in MTAP-positive and MTAP-negative NSCLC, pancreatic cancer and mesotheliomas cancer cell lines; and tested the anti-neoplastic efficacy of the combination of SDX-102 with docetaxel or 5-FU in vitro and using in vivo human xenograft models.

The effect of docetaxel or 5-FU, alone or in combination with L-alanosine, on proliferation and survival in MTAP-negative cells was measured by MTT assay 72 hours post-treatment. Docetaxol and 5-FU, when used alone, displayed a range of activity in several MTAP-negative cell lines comparable to the IC50s values reported in the literature for MTAP-positive cells from the same tumor type. Table 6 shows results of single agent activity of SDX-102, 5-FU, and docetaxel in selected pancreatic cancer, NSCLC, and mesothelioma cell lines.

TABLE 6
Effects of Single Drug Treatment

| | | IC₅₀ SDX- | IC₅₀ 5-FU | IC₅₀ docetaxel |
|---|---|---|---|---|
| Cell Line | Type | 102 (μM) | (μM) | (μM) |
| A549 | NSCLC | 0.3 ± 0.1 | 1 ± 0.6 | 5 ± 4 |
| NCI-H292 | NSCLC | 0.2 ± 0.06 | 3 ± 2 | 0.2 ± 0.05 |
| NCI-H322M | NSCLC | 6 ± 1 | 6 ± 5 | 6 ± 2 |
| PANC-1 | Pancreatic | 10 ± 5 | 14 ± 4 | 30 ± 6 |
| BX-PC3 | Pancreatic | 3 ± 1 | 3 ± 2 | 1.2 ± 0.8 |
| NCI-H2452 | Mesothelioma | 1 ± 0.5 | 5 ± 2 | 3 ± 2 |

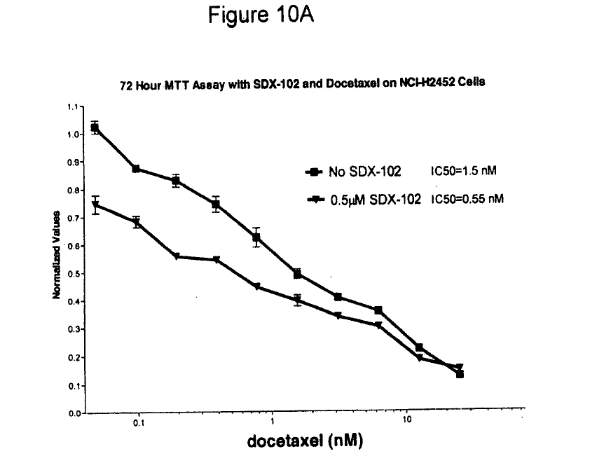

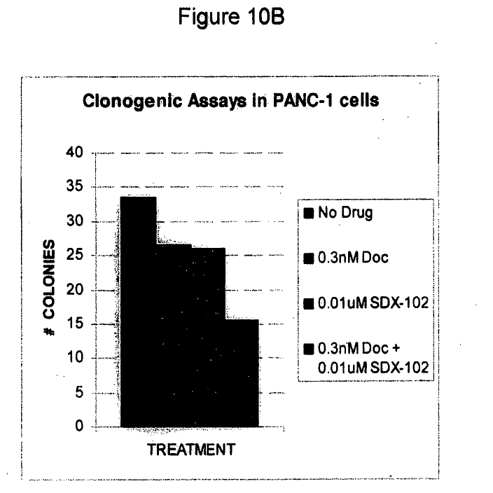

In vitro combinations of SDX-102 with 5-FU or docetaxel in MTAP-negative cells demonstrated additive to synergistic interactions when analyzed using the combinatorial index (CI) analysis method of Chou and Talalay (CI range: 1.1-0.5). Cells were incubated with various doses of docetaxel with or without 0.5 μM of SDX-102 for three days. For combinatorial index experiments, a constant ratio of both drugs were used. Cytotoxicity was measured using either MTT or MTS analysis. Drug combination effects were characterized based upon recommendations of the Combinatory Index Method as described by Chou and Talalay using the CalcuSyn Software for Dose Effect Analysis (Biosoft, Cambridge, U.K.). Combinations that showed additivity or synergism at the EC₇₅ are presented in Table 7. The analysis was repeated at least two times with similar results. Clonogenic or colony formation assays were performed in 6 well dishes using PANC-1 cells. Colonies were allowed to grow for approximately one month before they were stained and analyzed on an El Logic 200 Imaging System (Kodak) using Kodak ID Image Analysis Software to determine the number of colonies. Similar results were observed when comparing the size of the colonies generated (FIGS. 10A and 10B).

TABLE 7
Synergistic Effects of a Docetaxel/L-alanosine Combination

| Cell Line | Type | EC75 CI value | Effect |
|---|---|---|---|
| A549 | NSCLC | 0.8 | synergism |
| NCI-H292 | NSCLC | 1.1 | additive |
| NCI-H322M | NSCLC | 0.9 | synergism |
| PANC-1 | Pancreatic | 0.6 | synergism |
| NCI-H2452 | Mesothelioma | 0.7 | synergism |

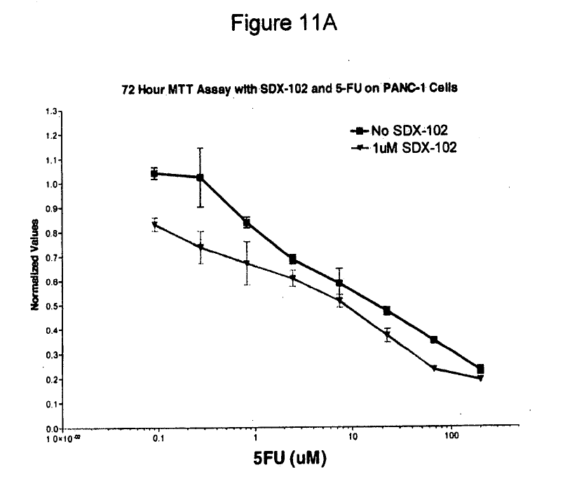

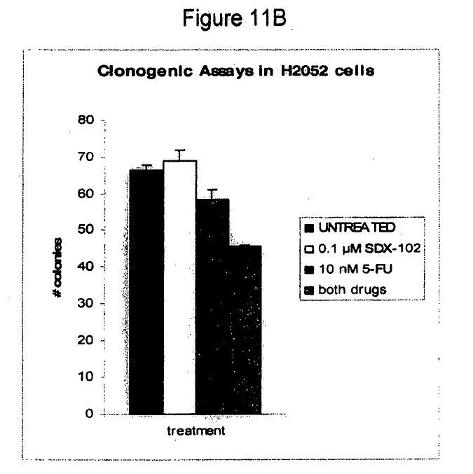

Cells were incubated with various doses of 5-FU with or without 0.5 μM of SDX-102 for three days. For combinatorial index experiments, constant ratios of both drugs were used. Cytotoxicity was measured using either MTT or MTS analysis. Drug combination effects were characterized based upon recommendations of the Combinatory Index Method as described by Chou and Talalay using the CalcuSyn Software for Dose Effect Analysis (Biosoft, Cambridge, U.K.). Combinations that showed additivity or synergism at the EC₇₅ are presented in Table 8. Analysis was repeated at least two times with similar results. Clonogenic or colony formation assays were performed in 6 well dishes using NCI-H2052 cells. Colonies were allowed to grow for approximately one month before they were stained and analyzed on an El Logic 200 Imaging System (Kodak) using Kodak ID Image Analysis Software to determine the number of colonies (FIGS. 11A and 11B).

TABLE 8
Synergistic Effects of a 5 FU/L-alanosine Combination

| Cell Line | Type | EC75 CI value | Effect |
|---|---|---|---|
| PANC-1 | Pancreatic | 0.6 | synergism |
| BX-PC3 | Pancreatic | 0.8 | synergism |
| NCI-H2452 | Mesothelioma | 0.5 | synergism |

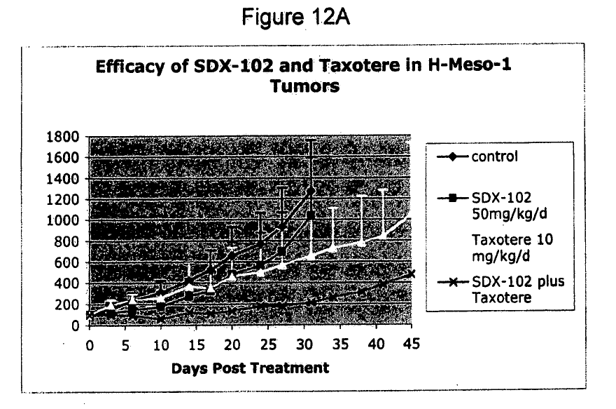

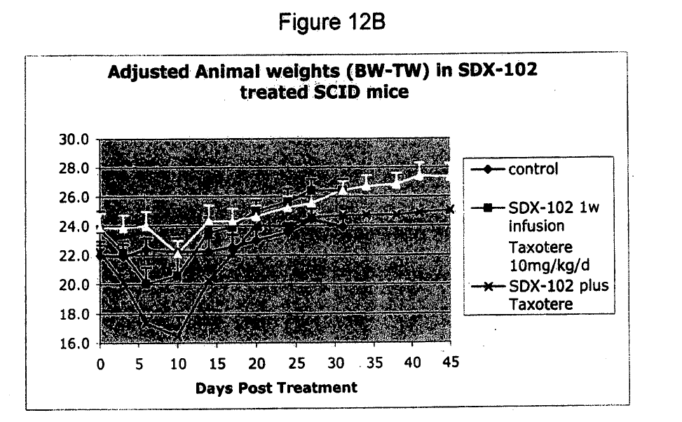

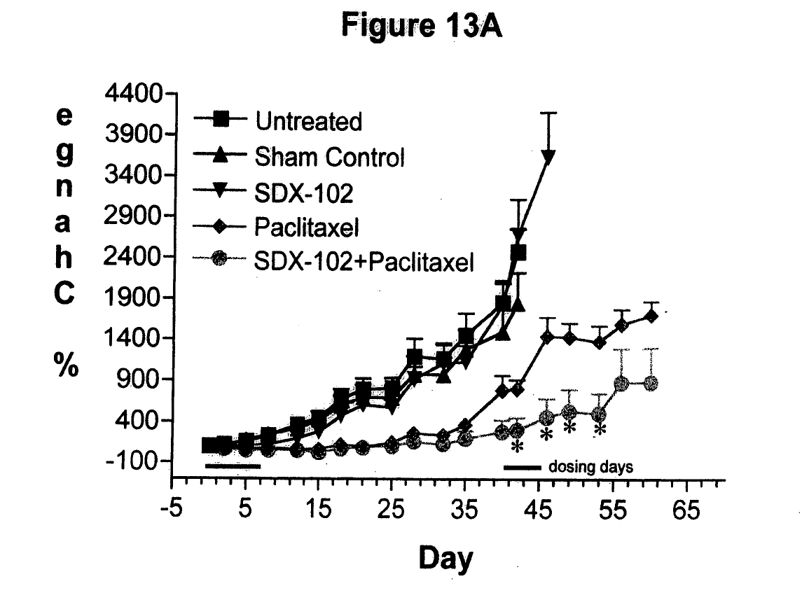

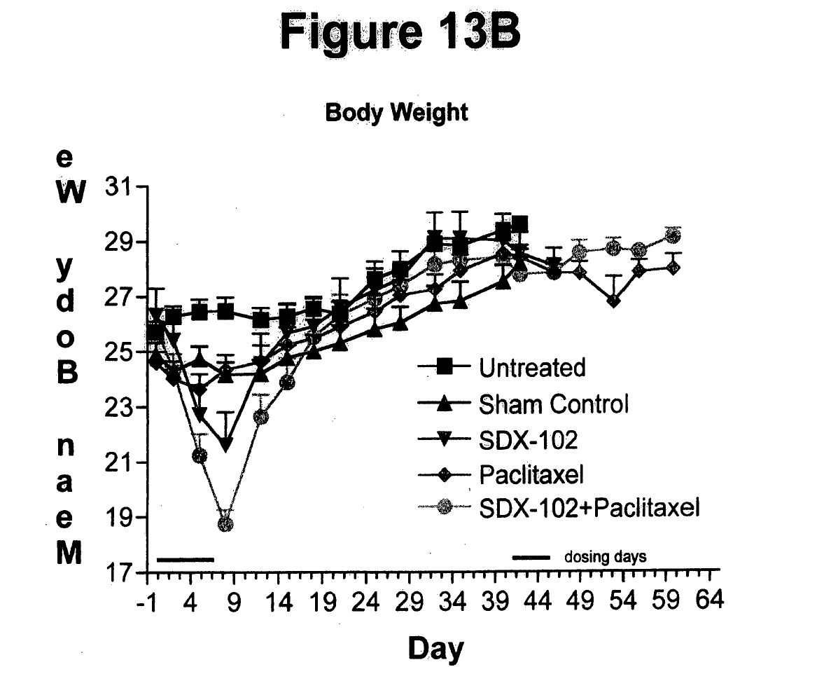

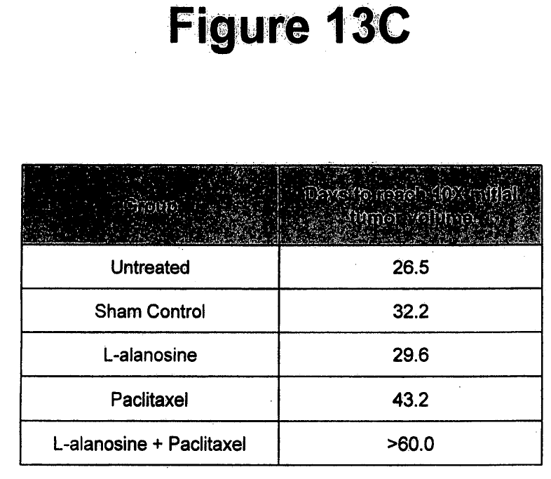

In vivo, the combination of docetaxel (10 mg/kg) and SDX-102 (50 mg/kg) was superior to either single agent in a mesothelioma xenograft model (H-Meso-1) in SCID mice. Treatment was initiated at a tumor volume of 100 mm³. Thirty-one days following treatment the mean tumor volume of the combination group was 217 mm³ compared to 655 mm³ and 1035 mm³ for taxotere and SDX-102-treated groups, respectively, while control tumors were 1272 mm³. SCID mice, 6-8 weeks old were inoculated subcutaneously with H-Meso-1 cells (1×106/mouse) and the treatment was initiated at a mean tumor volume of 100 mm³. SDX-102 was administered via an osmotic pump and Taxotere was administered intraperitoneally daily (Monday through Friday). Tumor volume and body weight were monitored twice weekly. Tumor volume was calculated by the formula 4/3 πr³. FIG. 12A shows tumor volume (mm³) over time and the FIG. 12B shows body weight (g) (corrected for tumor volume) over time.

In conclusion, L-alanosine displayed anti-tumor activity in NSCLC, mesothelioma, and pancreatic cancer cell lines. L-alanosine in combination with 5-FU or docetaxel resulted in a supra-additive response in vitro. In H-Meso-1 tumor xenografts in mice, the combination of L-alanosine with docetaxel was superior to either drug alone in inhibiting tumor growth. These results demonstrate that L-alanosine possesses anti-tumor activity and should be synergistic in combination with other anticancer drugs in vitro and in vivo.

### Example 5

### Effects of L-Alanosine in Combination with Paclitaxel

This example describes the results of a combination therapy of L-alanosine and paclitaxel (Taxol®)) in a mouse model of an MTAP-negative NSCLC tumor. As described above, the antitumor activity of L-alanosine, an amino acid analog inhibiting de novo adenylate synthesis, is potentiated by deficiency of methylthioadenosine phosphorylase (MTAP), an enzyme responsible for the salvage of adenine back into the adenylate pool. This example demonstrates the activity of L-alanosine and Taxol®0 (paclitaxel) in MTAP-negative A549 NSCLC xenografts. The A549 NSCLC cell line used in this study was obtained from the American Type Culture Collection (ATCC number CCL-185), and was passaged in culture using RPMI supplemented with 10% FBS. The MTAP status of the A549 NSCLC cells was determined by immunoblot using a monoclonal antibody, as described above.

Here, A549 cells (5×10⁶ cells in 200 μL per/mouse) were inoculated subcutaneously into the flanks of male SCID mice (each 6-8 weeks of age, obtained from Simonsen Laboratories, Inc. (Gilroy, Calif.)). After the tumors reached a volume of approximately 80-100 mm³, the mice were randomized into groups and treatment was initiated (day 0). Mice were treated with L-alanosine (prepared as a 167 mg/mL solution by dissolving L-alanosine in saline, and administered by subcutaneous infusion supplied by an implanted Alzet osmotic pump) or Taxol® (16 mg/kg/day, administered intraperitoneally in a 4 mg/mL drug-containing solution), or both (same respective doses and routes). Each chemotherapeutic compound (whether administered as a monotherapy or as part of a combination therapy) was administered in two cycles, with the first cycle beginning on day 0, and the second cycle beginning on day 41. In the first L-alanosine cycle, which lasted seven days, L-alanosine was delivered at the rate of 40 mg/kg/day, while the second cycle lasted five days and a smaller amount of L-alanosine (20 mg/kg/d) was administered by intraperitoneal injection. Each cycle in the paclitaxel treatment regimen lasted five days.

Tumor growth was monitored for 60 days, with tumor growth being assessed twice weekly by way of body weight and tumor volume measurements. Tumor volume was determined by measuring the tumors in three dimensions, with volume being calculated using the formula: 4/3r³.

Over the course of the study, the L-alanosine monotherapy regimen did not induce tumor growth inhibition at the doses administered. On the other hand, paclitaxel treatment, alone or in combination with an L-alanosine treatment regimen, inhibited tumor growth in all treatment groups, with the combination regimen being the most effective. See FIGS. 13A-C. The time for the tumors to increase 10-fold in size (i.e., to a volume of about 1000 mm³) from the initial mean volume (about 1000 mm³) were 32.2, 29.6, 43.2, and more than 60 days for the control, L-alanosine, paclitaxel, and the combination group, respectively. On day 46, the statistically significant percent mean change in tumor volume for the paclitaxel-alone and the combination treatment group was 1449±236 and 464±225, respectively (P=0.01, unpaired t-test). In addition, three out of eight mice remained tumor-free (non-palpable) in the combination treatment group at the end of the study (day 60). There were no tumor-free mice in any of the other groups. These results show that L-alanosine enhances the anti-tumor activity of paclitaxel when administered in vivo as part of a combination therapy.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the spirit and scope of the invention. More specifically, it will be apparent that certain agents which are both chemically and physiologically related to alanosine may be substituted for the agents described herein while the same or similar results would be achieved. All such similar substitutes and modifications apparent to those skilled in the art are deemed to be within the spirit and scope of the invention as defined by the appended claims.

All patents, patent applications, and publications mentioned in the specification are indicative of the levels of those of ordinary skill in the art to which the invention pertains. All patents, patent applications, and publications are herein incorporated by reference in their entirety for all purposes and to the same extent as if each individual publication was specifically and individually indicated to be incorporated by reference.

The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A composition comprising alanosine and a solvent, wherein the pH of the composition is at least about 7.5 and the alanosine is stable for at least about one month when stored as a liquid.

2. A composition according to claim 1 wherein the alanosine is selected from the group consisting of L-alanosine and D-alanosine.

3. A composition according to claim 1 wherein the pH is within the range of about 8 to about 12.

4. A composition according to claim 1 wherein the pH is selected from the group consisting of a pH of about 8.5 and more than about pH 9.

5. A composition according to claim 1 that is stored in a pH-insensitive container.

6. A composition according to claim 5 wherein the pH-insensitive container is comprised of a material selected from the group consisting of glass and plastic.

7. A composition according to claim 1 wherein the composition, when stored as a liquid, is stable for a period selected from the group consisting of at least about six months and at least about twelve months.

8. A composition according to claim 1 wherein less than about 1% of the alanosine becomes degraded after about 24 months of storage.

9. A composition according to claim 8 wherein storage is at a temperature selected from the group consisting of room temperature and about 5° C.

10. A composition according to claim 1 that is pharmaceutically acceptable.

11. A composition comprising L-alanosine, water for injection, and a pH of about 8.5, wherein the L-alanosine is stable for at least about one year when stored as a liquid in a pH-insensitive container.

12. A method of making a stable aqueous formulation of alanosine, comprising:
a. dissolving or dispersing alanosine molecules in water to make an alanosine solution or suspension, wherein the alanosine molecules are selected from the group consisting of an alanosine acid salt and an alanosine base salt;
b. adjusting the pH of the solution or suspension to at least about 7.5; and
c. after adjusting the pH, storing the resulting solution in a pharmaceutically acceptable container.

13. A method according to claim 12 wherein the pharmaceutically acceptable container is pH-insensitive.

14. A method of treating a patient having a disease susceptible to treatment with alanosine, comprising administering to the patient a composition according to claim 1.

15. A method according to claim 14 wherein the patient is human and the disease is a cancer characterized by tumor cells that are MTAP deficient.

16. A method according to claim 15 wherein the cancer is selected from the group consisting of an acute lymphoblastic lymphoma, a glioma, a non-small cell lung cancer, a urothelial tumor, non-Hodgkins lymphoma, mesothelioma, leukemia, bladder cancer, pancreatic cancer, soft tissue sarcoma, osteosarcoma, head and neck cancer, and myxoid chondrosarcoma.

17. A method according to claim 15 further comprising administering to the patient a therapeutically effective amount of a second chemotherapeutic agent.

18. A method according to claim 17 wherein the second chemotherapeutic agent is selected from the group consisting of docetaxel, 5-fluorouracil, vinorelbine, pemetrexed, gemcitabine, erlotinib, gefitinib, and paclitaxel.

19. A kit comprising a composition according to claim 1 stored in a container.

20. A method of treating a patient having a disease susceptible to treatment with alanosine, comprising administering to the patient a first composition comprising a therapeutically effective amount of L-alanosine and a second composition comprising a therapeutically effective amount of a second chemotherapeutic agent.
